(19) United States
(12) Patent Application Publication
(10) Pub. No.: US 2024/0207064 A1
(43) Pub. Date: Jun. 27, 2024

(54) SURGICAL IMPLANT DEVICE INCORPORATING A LATTICE VOLUME AND ASSOCIATED METHOD OF MANUFACTURE

(71) Applicant: Kyocera Medical Technologies, Inc., Redlands, CA (US)

(72) Inventors: Scott Alan Rucker, Austin, TX (US); Joseph Virgil Thompson, Round Rock, TX (US); Trace Cawley, Austin, TX (US); Dennis Ty, Franklin, TN (US)

(21) Appl. No.: 18/599,523

(22) Filed: Mar. 8, 2024

Related U.S. Application Data
(63) Continuation-in-part of application No. 17/072,111, filed on Oct. 16, 2020.
(60) Provisional application No. 63/451,082, filed on Mar. 9, 2023.

(51) Int. Cl. A61F 2/44; A61F 2/30

(52) U.S. Cl. CPC A61F 2/4455; A61F 2002/30321; A61F 2002/30593; A61F 2002/30787; A61F 2002/3092

(57) ABSTRACT

A surgical implant device includes opposing first and second solid surfaces and a third solid surface connecting the first solid surface and the second solid surface. The third solid surface includes a hole penetrating through the first and second solid surfaces. The hole includes an outer surface with a first region and an inner surface. A surface roughness of the first region is greater than a surface roughness of the inner surface.

### CROSS-REFERENCE TO RELATED APPLICATIONS

This patent application is a continuation-in-part of U.S. patent application Ser. No. 17/072,111, filed Oct. 16, 2020. This application also claims the benefit of U.S. Provisional Application No. 63/451,082, filed Mar. 9, 2023. Each of these patent applications are incorporated by reference herein.

### TECHNICAL FIELD

The present disclosure relates generally to the surgical and medical device fields. More particularly, the present disclosure relates to a surgical implant device incorporating a lattice volume and an associated method of manufacture.

### BACKGROUND

A variety of conventional surgical implant devices, such as spinal and other orthopedic implant devices, exist that incorporate internal voids that are intended to impart such surgical implant devices with a degree of elasticity and provide areas for bone graft placement and bony ingrowth and purchase, while attempting to maintain structural integrity and strength, especially when such surgical implant devices are manufactured from metallic and/or polymeric materials. Weight savings may also be a consideration in some applications. These internal voids may take the form of discrete holes and/or pores, strut assemblies, and/or lattice volumes, for example. However, to date, such conventional surgical implant devices do not perform adequately and/or are difficult to manufacture.

### SUMMARY

In various illustrative embodiments, the present disclosure provides a surgical implant device, such as a spinal or other orthopedic implant device, that incorporates both solid surfaces and an internal lattice volume. Specifically, an anterior lumbar interbody fusion (ALIF) cage is provided as an example. In a cervical spine embodiment, a cage for the cervical spine is provided. This internal lattice volume utilizes more numerous, smaller pores and fine struts adjacent to the solid surfaces and less numerous, larger pores and thicker struts remote from the solid surfaces, thereby providing superior elasticity, bony ingrowth and purchase, and structural integrity and strength properties. Conventional internal voids and the like may also be provided for bone graft placement, etc. The surgical implant device of the present disclosure is developed using a computer-aided design (CAD) model and manufactured from a metallic (e.g., titanium) or polymeric (e.g., poly ether ether ketone (PEEK)) material using an additive manufacturing process, such as three-dimensional (3D) printing, or a more traditional manufacturing process.

In one illustrative embodiment, the present disclosure provides a surgical implant device, including: a solid surface; and a lattice structure disposed adjacent to the solid surface, wherein the lattice structure includes a first plurality of struts that define a first plurality of voids adjacent to the solid surface and a second plurality of struts that define a second plurality of voids remote from the solid surface. Each of the first plurality of struts has an average cross-sectional diameter that is smaller than an average cross-sectional diameter of each of the second plurality of struts. Each of the first plurality of voids has an average internal diameter that is smaller than an average internal diameter of each of the second plurality of voids. The first plurality of struts and the first plurality of voids have an overall material density that is approximately equal to the second plurality of struts and the second plurality of voids. Optionally, the lattice structure further includes a third plurality of struts that define a third plurality of voids disposed between and coupling the first plurality of struts and the first plurality of voids and/to the second plurality of struts and the second plurality of voids. Optionally, the solid surface is disposed at an external periphery of the surgical implant device. Alternatively, the solid surface is disposed at an internal portion of the surgical implant device. The solid surface and the lattice structure are integrally formed. The surgical implant device also includes a needle-populated porous surface disposed adjacent to the solid surface opposite the lattice structure. The solid surface and the needle-populated porous surface are integrally formed.

In another illustrative embodiment, the present disclosure provides a method for manufacturing a surgical implant device, including: designating a portion of a virtual volume as a solid surface; selecting a first plurality of points within the virtual volume adjacent to the solid surface that locate a first plurality of struts that define a first plurality of voids; selecting a second plurality of points within the virtual volume remote from the solid surface that locate a second plurality of struts that define a second plurality of voids; locating the first plurality of struts that define the first plurality of voids within the virtual volume using the first plurality of points; and locating the second plurality of struts that define the second plurality of voids within the virtual volume using the second plurality of points. The method also includes thickening each of the first plurality of struts and the second plurality of struts within the virtual volume such that each of the first plurality of struts has an average cross-sectional diameter that is smaller than an average cross-sectional diameter of each of the second plurality of struts. The method further includes thickening each of the first plurality of struts and the second plurality of struts within the virtual volume such that each of the first plurality of voids has an average internal diameter that is smaller than an average internal diameter of each of the second plurality of voids. The method still further includes thickening each of the first plurality of struts and the second plurality of struts within the virtual volume such that the first plurality of struts and the first plurality of voids have an overall material density that is approximately equal to the second plurality of struts and the second plurality of voids. Optionally, the solid surface is disposed at an external periphery of the virtual volume. Alternatively, the solid surface is disposed at an internal portion of the virtual volume. The method includes additively manufacturing the surgical implant device using the virtual volume including the designated solid surface and the located first plurality of struts that define the first plurality of voids and second plurality of struts that define the second plurality of voids. The method also includes defining a needle-populated porous surface adjacent to the solid surface. The method further includes additively manufacturing the solid surface and the needle-populated porous surface. The method includes additively manufacturing the solid surface and the needle-populated porous surface from one of a metallic material and a polymeric material.

### BRIEF DESCRIPTION OF THE DRAWINGS

The present disclosure is illustrated and described with reference to the various drawings, in which like reference numbers are used to denote like device components/method steps, as appropriate, and in which:

FIGS. 1A and 1B are examples of the use of the spinal implant of one illustrative embodiment of the present disclosure;

FIG. 2 is a perspective view of one illustrative embodiment of the implant device of the present disclosure;

FIGS. 3A and 3B are perspective views of one illustrative embodiment of the implant device of the present disclosure;

FIGS. 4A, 4B, 4C, 4D, 4E and 4F are perspective views of one illustrative embodiment of the implant device of the present disclosure, whereinFIGS. 4A and 4D are perspective views of one illustrative embodiment of the implant device of the present disclosure, FIGS. 4B and 4E are enlarged views of the dotted portion of FIGS. 4A and 4D, and FIGS. 4C and 4F are back views of the front solid surface of the implant device;

FIGS. 5A, 5B, 5C, 5D, 5E, 5F, 5G and 5H are side views of one illustrative embodiment of the implant device of the present disclosure, wherein FIGS. 5A and 5E are side views of the implant, FIGS. 5B and 5F are enlarged views of the dotted portion of FIG. 5, and FIGS. 5C and 5G are front views of the implant device;

FIG. 6 is schematic diagram illustrating the overall methodology of generating the implant device of FIG. 2;

FIG. 7 is schematic diagram illustrating a mesh generation step in the overall methodology of FIG. 6;

FIG. 8 is schematic diagram illustrating a first point selection step in the overall methodology of FIG. 6;

FIG. 9 is schematic diagram illustrating a second point selection step in the overall methodology of FIG. 6;

FIG. 10 is schematic diagram illustrating a lattice generation step in the overall methodology of FIG. 6;

FIG. 11 is schematic diagram illustrating a strut thickening step in the overall methodology of FIGS. 3A AND 3B;

FIG. 12 is a schematic diagram illustrating one example embodiment of the needle-populated porous structure that may cover the exterior of the implant device of FIG. 2;

FIG. 13 is a network diagram of a cloud-based system for implementing various cloud-based operations of the present disclosure;

FIG. 14 is a block diagram of a server which may be used in the cloud-based system of FIG. 13 or the like;

FIG. 15 is a block diagram of a user device which may be used in the cloudbased system of FIG. 13 or the like;

FIGS. 16A and 16B are perspective views of one illustrative embodiment of the implant device of the present disclosure;

FIGS. 17A and 17B are perspective view of one illustrative embodiment of the implant device of the present disclosure;

FIG. 18 is a schematic diagram of one illustrative embodiment of the implant device of the present disclosure; and

FIGS. 19A, 19B, 19C and 19D are side views of one illustrative embodiment of the implant device of the present disclosure, wherein FIG. 19B and 19D are side views of the implant with the additional dotted lines as imaginary lines that schematically show the parallel struts compared to FIGS. 19A and 19C.

### DESCRIPTION OF ILLUSTRATIVE EMBODIMENTS

The present disclosure provides a surgical implant device, such as a spinal or other orthopedic implant device, that incorporates both solid surfaces and an internal lattice volume. Specifically, an ALIF cage is provided as an example. This internal lattice volume utilizes more numerous, smaller pores and fine struts adjacent to the solid surfaces and less numerous, larger pores and thicker struts remote from the solid surfaces, thereby providing superior elasticity, bony ingrowth and purchase, and structural integrity and strength properties. Conventional internal voids and the like may also be provided for bone graft placement, etc. The surgical implant device of the present disclosure is developed using a CAD model and manufactured from a metallic (e.g., titanium) or polymeric (e.g., PEEK) material using an additive manufacturing process, such as 3D printing, or a more traditional manufacturing process.

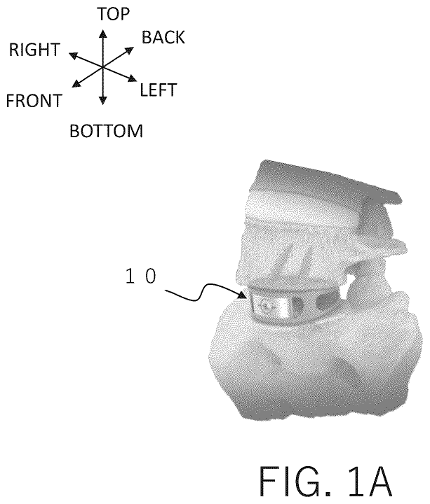

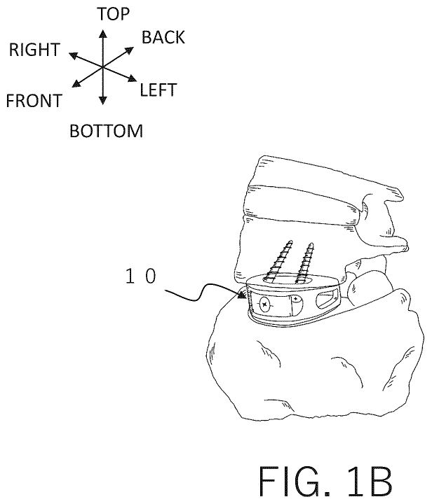

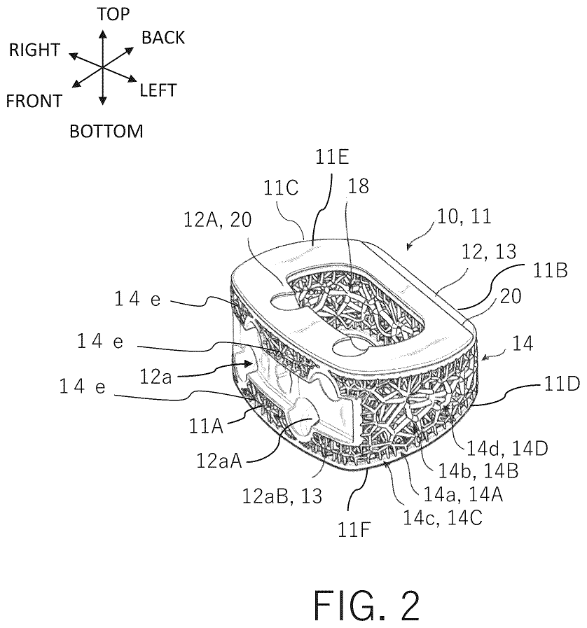

First, a mode of use of an implant device 10 will be described with reference to FIGS. 1A and 1B. Hereinafter, a direction indicated by an arrow denoted as “front”, “back”, “top”, “bottom”, “right” or “left” are referred to as “front side”, “back side”, “top side” “bottom side”, “right side” or “left side”. The front side in FIGS. 1A and 1B are ventral sides of a human body. As illustrated in FIG. 2, the implant device 10 according to the present embodiment is formed by a base 11 having a substantially elliptical column shape extending in a front-back direction, and includes a front face 11A, a back face 11B, a right side face 11C, a left side face 11D, a top face 11E, and a bottom face 11F.

For example, the implant device 10 has a substantially elliptical planar shape when viewed from the top side or bottom side. The implant device 10 has a void 18 in a region containing the center in a planar shape. The implant device 10 has a plurality of holes 12a in the front face 11A. The number of the holes 12a may be one.

The holes 12a are provided to penetrate from the front face 11A to the top face 11E or to the bottom face 11F. The holes 12a are also provided to penetrate from the front face 11A to the void 18. The holes 12a may be through-holes (unthreaded through-holes) which a spinal fixation bolt can pass through, or may be through-holes with threads. The implant device 10 may have a mixture of the holes 12a with threaded holes and the holes 12a with through-holes without threaded holes.

Referring now specifically to FIG. 2, in one illustrative embodiment, the implant device 10 of the present disclosure includes one or more solid surfaces 12. The implant device 10 includes one or more lattice volume 14. As used herein, “solid” means having a relatively higher density and/or lower porosity than the adjacent lattice volume 14. In other words, the density of the lattice volume 14 is low compared to the density of the solid surface 12, and the porosity of the lattice volume 14 is high compared to the porosity of the solid surface 12. The thickness of the solid surface 12 may be configured thicker than, for example, the thickness of the struts 14c or/and the struts 14d which are described below. Alternatively, the thickness of the solid surface 12 may be thinner than, for example, the struts 14c. Alternatively, the thickness of the solid surface 12 may be, for example, thicker than the struts 14c and thinner than the struts 14d.

The solid surfaces 12 may surround the entire exterior, or only portions of the exterior, of the implant device 10. The solid surface 12 may be, for example, an intermediate device surface and/or may be located on the inner periphery of the implant device 10. The solid surface 12 may be located at least in one of the front face 11A, the back face 11B, the right side face 11C, the left side face 11D, the top face 11E and the bottom face 11F of the implant device 10. The solid surface 12 may also be located on at least a portion of the surface of the void 18. Here, the perimeter refers to the outer perimeter of the implant device 10, including, for example, the front face 11A, the back face 11B, the right side face 11C, the left side face 11D, the top face 11E, and the bottom face 11F. Also, the inner perimeter refers to the inner perimeter of the implant device 10, including, for example, the surface of the void 18.

As shown in FIG. 2, the implant device 10 has the solid surface 12 on the top, bottom and front sides. The implant device 10 is configured such that the solid surface 12 of the front side is continuously connected to the solid surfaces 12 of top side and bottom side. With this configuration, the implant device 10 ensures stiffness. The implant device 10 also has a bone-contact surface 12A in contact with bone located on the solid surface 12 of the top face 11E and/or the bottom face 11F.

The Implant device 10 Ides the lattice volume 14 disposed adjacent to, or between, the solid surfaces 12. The lattice volume 14 includes a plurality of struts that define a plurality of pores. The lattice volume 14 may be located at least on the outer periphery, on inner periphery, and inside of the implant device 10. For example, the lattice volume 14 is exposed on the outer and inner periphery of the implant device 10.

The lattice volume 14 defines more numerous, smaller pores 14a adjacent to the solid surfaces 12 and less numerous, larger pores 14b remote from the solid surfaces 12. Correspondingly, the lattice volume 14 utilizes more numerous, finer struts 14c adjacent to the solid surfaces 12 and less numerous, thicker struts 14d remote from the solid surfaces 12.

In other words, the lattice volume 14 has a plurality of first struts 14C defining a plurality of first pores 14A adjacent to the solid surface 12. The lattice volume 14 also has a plurality of second struts 14D defining a plurality of second pores 14B away from the solid surface 12. The plurality of first pores 14A have at least two or more pores 14a. The plurality of second pores 14B have at least two or more pores 14b. The plurality of first struts 14C have at least two or more struts 14c. The plurality of second struts 14D have at least two or more struts 14d.

The cross-sectional areas in the longitudinal direction of the struts 14c are smaller than the cross-sectional area in the longitudinal direction of the struts 14d. The pores 14a are smaller than the pores 14b. Specifically, when the pores are defined as areas surrounded by struts, it is sufficient that one volume of a given pore 14a is smaller than one volume of a given pores 14b. When considered in two dimensions, the volumes of the pores 14a and 14b may be, for example, the area enclosed by the struts.

Here, the plurality of first struts 14C may be located between the solid surface 12 and the plurality of second struts 14D. The solid surface 12, the plurality of first struts 14C, and the plurality of second struts 14D may be arranged in that order. In other words, the plurality of second struts 14D may contact the solid surface 12 through the plurality of first struts 14C. The plurality of second struts 14D may be positioned so as to be surrounded by the plurality of first struts 14C.

The plurality of first struts 14C may be located at one or more locations in the implant device 10. The plurality of second struts 14D may be placed at one or more locations in the implant device 10. The plurality of first struts 14C or/and the plurality of second struts 14D may be positioned, for example, behind the solid surface 12 of the front side. Alternatively, the plurality of first struts 14C or/and the plurality of second struts 14D may be located between the solid surface 12 of top side or/and the solid surface 12 of bottom side and the solid surface 12 of the front side.

In FIG. 2, the plurality of first struts 14C are positioned adjacent to the solid surface 12 of front side. Also, the plurality of first struts 14C are arranged adjacent to the solid surface 12 of top side. In addition, the plurality of first struts 14C are positioned adjacent to the solid surface 12 of bottom side. The first plurality of struts 14C are located between the solid surface 12 and the plurality of second struts 14D. In other words, the plurality of second struts 14D are arranged to be surrounded by, for example, the plurality of first struts 14C positioned adjacent to the solid surface 12 of front side, the plurality of first struts 14C adjacent to the solid surface 12 of top side, and the plurality of first struts 14C adjacent to the solid surface 12 of bottom side.

The pores 14a, 14b that the lattice volume 14 can have regular or random shapes, dimensions, and/or volumes. The struts 14c, 14d that the lattice volume 14 can have a regular or random cross-sectional shape, length, and/or diameter.

The thickness of the struts 14c may be, for example, about 0.45 mm. The thickness of the struts 14c may be, for example, about 1 mm. It will be readily apparent to those skilled in the art that other dimensions can be used as well. The thickness of the struts 14c or/and the struts 14d can vary along the length of the struts. For example, the thickness of the struts 14c or/and the struts 14d may be thinner or thicker as the struts approach the solid surface 12. Alternatively, the thickness of the struts 14c and 14d along their length may be configured to have the maximum thickness between the points connecting the supports.

The plurality of first strut” 14C′and the plurality of first pores 14A can have an overall material density that is approximately equal to an overall material density of the plurality of second struts 14D and the plurality of second pores14B. The material density may be, for example, 80% to 100%, but it may be 60% to 80%. It will be readily apparent to those skilled in the art that other porosities can be used as well.

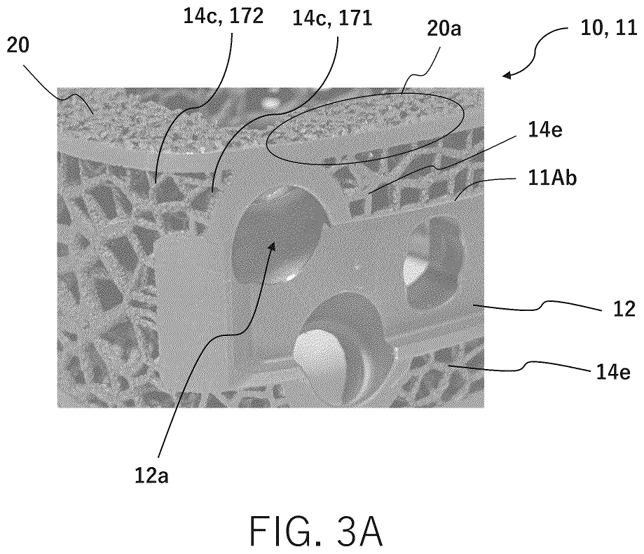

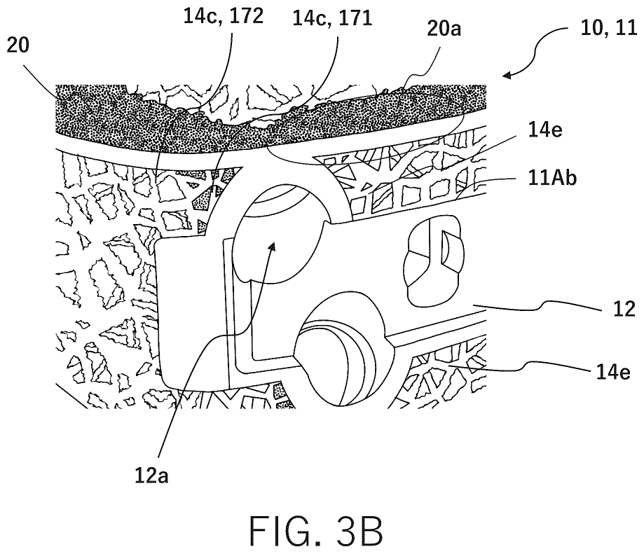

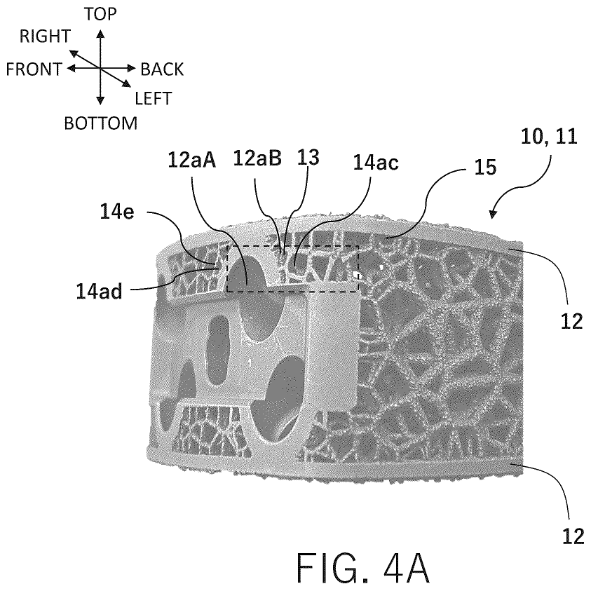

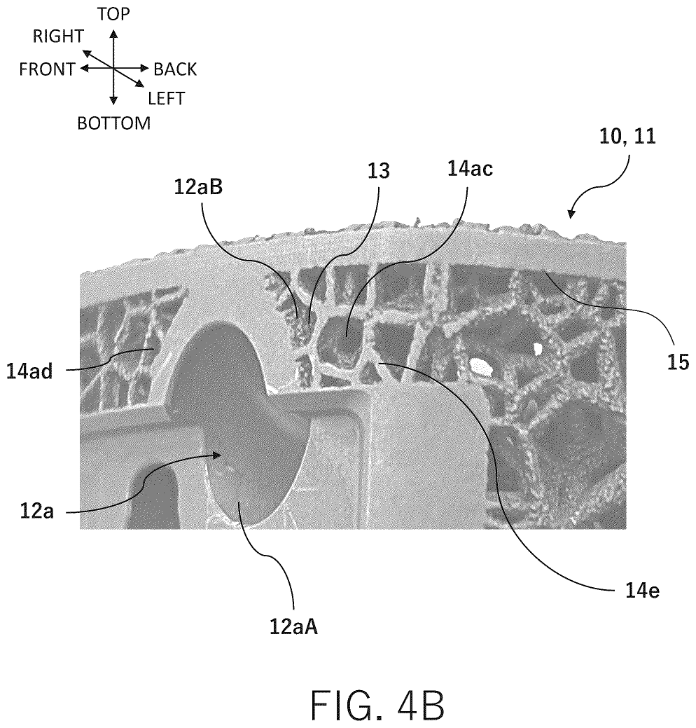

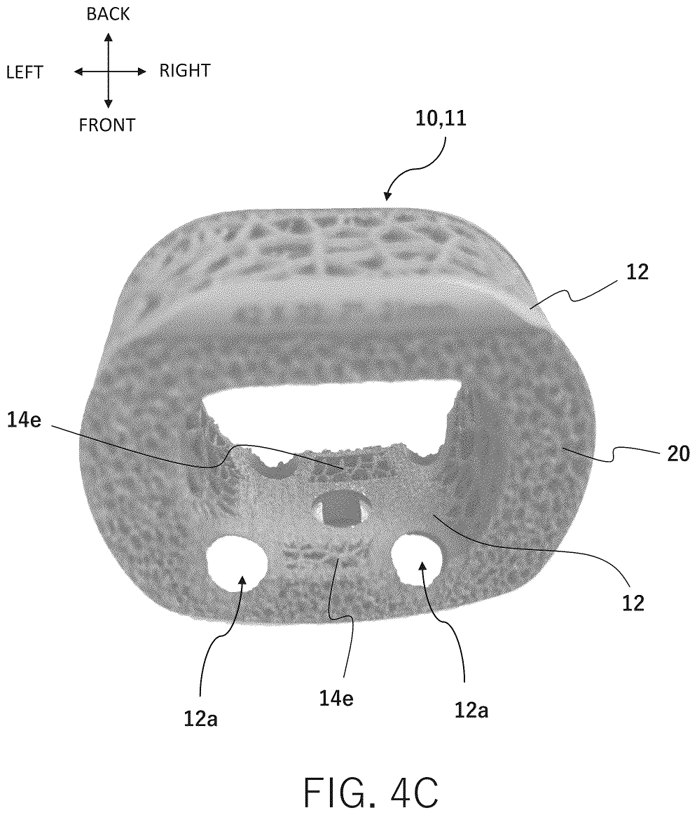

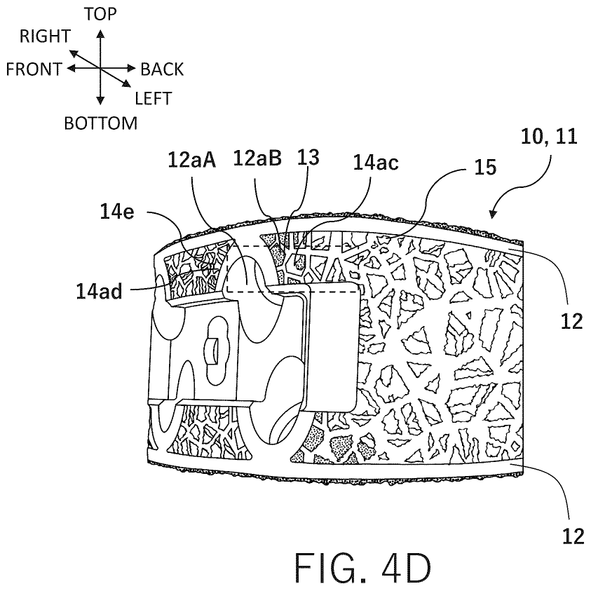

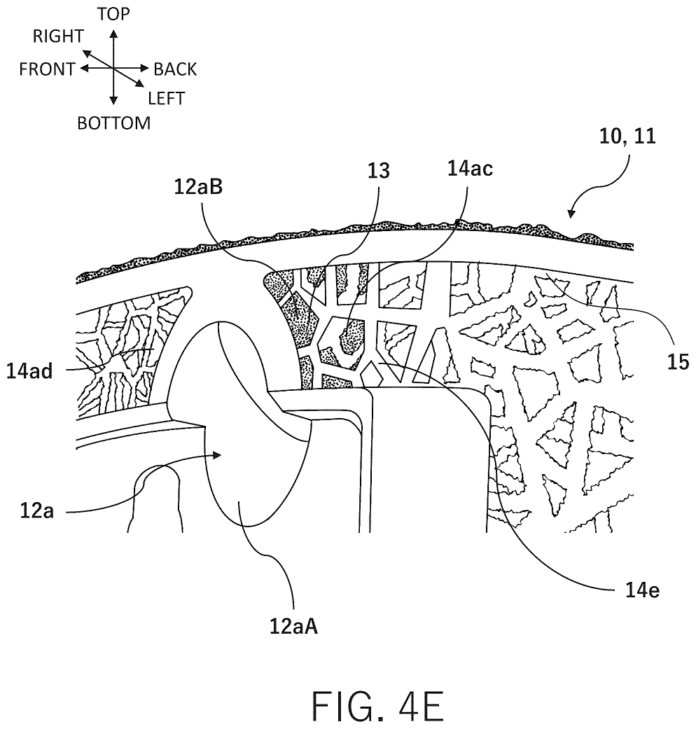

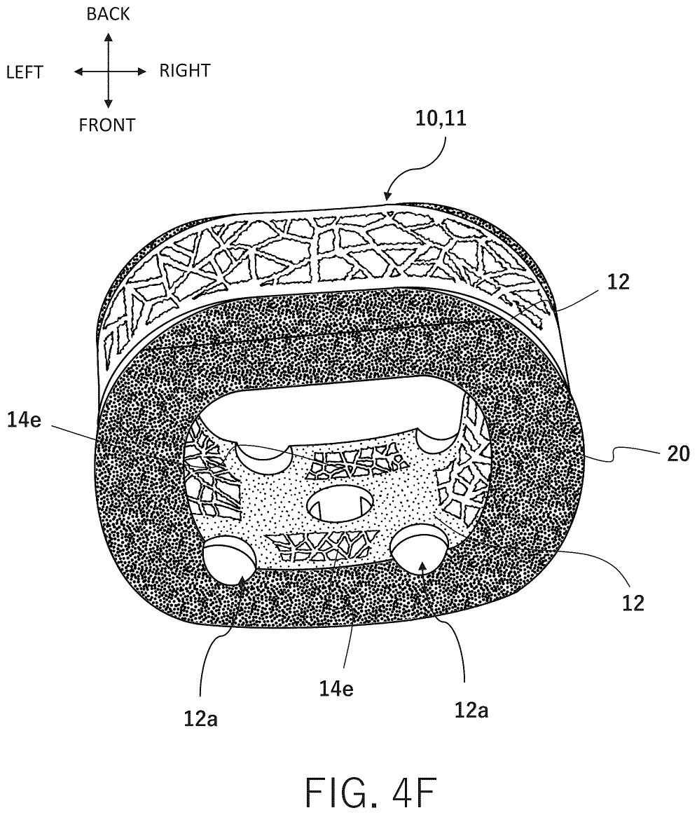

The implant device 10 has struts 14e positioned on thefront side as shown in FIGS. 3, 4A 4B and 4C. The struts 14e are located, for example, between the solid surface 12 of top side and the solid surface 12 of front side or/and between the solid surface 12 of lower side and the solid surface 12 of front side. The thickness of the struts 14e are set to be thinner than, for example, the thickness of the struts 14d. In other embodiments, the thickness of struts 14e may be, for example, the same as the thickness of the struts 14c and/or the struts 14d. The struts 14e may have a mixture of struts with different thicknesses.

Some of the frontmost struts in the struts 14e may be of a different shape than other struts (including the struts 14c and 14d). More specifically, the frontmost struts in the struts 14e may have flat portions compared to the struts located inside the frontmost struts in the struts 14e (for example, the struts may be the struts 14c or 14d). In other words, the struts 14e located on an outer surface may have one or more flat portions compared to struts that are not located on an outer surface.

The struts 14e with the flat portions may be all of the frontmost struts in the struts 14e or may be part of the frontmost struts in the struts 14e. The flat portions of the struts 14e may, for example, be coplanar with a face constituting the hole 12a or/and the front face of the solid surface 12. The p“rase “be coplana” with” means that a first plane including an area of a predetermined range and a second plane including an area different from the area of the predetermined range constitute a same plane.

The surface roughness of the flat portions of the struts 14e may be less than, for example, the surface roughness of the struts 14c or/and the struts 14d. Alternatively, the surface roughness of the flat portions of the struts 14e may be smaller than a surface roughness of the non-flat portions of the same struts 14e.

The flat portions of the struts 14e can be flattened together, for example, when polishing the front face 11A of the solid surface 12. Similar to the struts 14e, the struts 14c and/or the struts 14d may have flat portions at the outermost or/and innermost (cavity 18 side) struts.

The struts 14e may be loIated inside the edge 11Ab of the front face 11A of the solid surface 12 of front side as shown in FIGS. 3A and 3B. In the implant device 10, the front of the holes 12a is positioned inside the front of the front face 11A of the solid surface 12. The front of the holes 12a is coplanar with the flat portion of the struts 14e.

Cross-sectional shapes of the struts 14c, 14d and 14e cut in directions perpendicular to the length direction may have any suitable shape. The struts 14c, 14d and 14e may be, for example, circular, elliptical, triangular, square, rectangular, pentagonal, octagonal, irregular shapes and combinations thereof. The struts 14c may be approximately perpendicular to the solid surface 12.

One or more of the solid surfaces 12 may include a porous surface 20 positioned opposite to the lattice volume 14. The porous surface 20 may be located on a bone contacting surface 12A that will be described in detail later. The porous surface 20 may, for example, have a shape mimicking cancellous bone. More specifically, the width, depth or spacing of ruggedness of the porous surface 20 may smaller than the gap of cancellous bone or larger than the gap of cancellous bone by 10% to 20%.

The porous surface 20 may be located on the surface opposite to the lattice volume 14 (outer surface) at the solid surfaces 12 of the top and/or bottom side. The porous surface 20 may contain, for example, a collection of at least fifty, a hundred, two hundred, five-hundred or more needles. Such needles 28 substantially increase the coefficient of friction of the implant surface. Having a high coefficient of friction is clinically advantageous because it provides stronger initial fixation, which is important before bone is able to grow onto/into the porous structure 20.

The porous surfaces 20 may have flat regions 20a that have flattened edges, as shown in FIGS. 3A and 3B. The flat regions 20a may, for example, constitute surfaces inclined inwardly to the sides of the solid surfaces 12. In this case, the flat regions 20a may be provided by tilting and flattening the sides of the solid surfaces 12. Without limitation, the flat regions 20a may be coplanar with, for example, the sides of the solid surfaces 12. In this case, the porous surfaces 20 may be flattened so that the flat regions 20a becomes coplanar with the sides of the solid surfaces 12 when the sides of the solid surfaces 12 are flattened.

As shown in FIGS. 4A, 4B, 4D and 4E, the hole 12a of the solid surface 12 of front side has an inner surface 12aA and an outer surface 12aB. The hole 12a may have a first region 13 where the surface roughness of the outer surface 12aB is large compared to that of the inner surface 12aA.

The surface roughness of the inner surface 12aA is less than the surface roughness of the outer surface 12aB. For example, the surface roughness of the inner surface 12aA may be made smaller than the surface roughness of the outer surface 12aB by being polished after the hole 12a is formed. Alternatively, when the implant device 10 is manufactured by, for example, 3D printing, it may be designed and/or manufactured so that the surface roughness of the inner surface 12aA is greater than the surface roughness of the outer surface 12aB.

The outer surface 12aB may have the first region 13 with appropriate surface roughness. The surface roughness of the first region 13 may be greater than, for example, the surface roughness of at least one of the struts 14c, 14d and 14e. Alternatively, the surface roughness of the first region 13 may be, for example, greater than the surface roughness of the inner surface 12aA and smaller than the surface roughness of at least one of the struts 14c, 14d and 14e. In addition, the surface roughness of the first region 13 may be smaller than, for example, the surface roughness of the porous surface 20.

The first region 13 may be located, for example, throughout the outer surface 12aB.

In addition, the first region 13 may be located on a part of the outer surface 12aB. The outer surface 12aB may continuously connect to at least one of struts 14c, 14d and 14e.

The hole 12a has the first region 13 on the outer surface 12aB, which may be suitable for fixing the implant device 10 in the body. The implant device 10 may easily utilize the outer surface 12aB as a scaffold for bone ingrowth, while reducing the possibility of a portion of the inner surface 12aA chipping away to produce metal powder.

Examples of surface roughness indices include arithmetic mean roughness Sa (ISO 25178), but it is not limited to this. The first region 13 may include places where the surface roughness is locally greater than the surface roughness of the plurality of the struts, and the outer surface 12aB may include places where the surface roughness is locally less than that the surface roughness of the first region 13.

Surface roughness may be measured in a stylus or optical format. Surface roughness may be measured according to, for example, ISO 25178. Surface roughness measurement is not limited to the methods described above. For example, the surface roughness may be calculated from an image taken of a given section by an optical or electron microscope, etc. Any section can be obtained, for example, by cutting or polishing a resin filled implant device 10. When comparing surface roughness, for example, surface roughness measured by the same method may be used, or surface roughness measured by different methods may be used.

As shown in FIGS. 4A, 4B, 4D and 4E, the lower surface of the solid surface 12 of the top side may include a second region 15 which surface roughness is greater than the surface roughness of the inner surface 12aA of the hole 12a. The struts side surface of the solid surface 12 may have the second region 15 with appropriate surface roughness. The surface roughness of the second region 15 may be greater than, for example, the surface roughness of at least one of the struts 14c, 14d and 14e. The surface roughness of the second region 15 may be smaller than the surface roughness of the porous surface 20.

More specifically, the second region 15 may be located below the solid surface 12 of the top side or/and above the solid surface 12 of the bottom side. Alternatively, the second region 15 may be located behind the solid surface 12 of the front side. Since the struts side surface of the solid surface 12 is the second region 15, the second region 15 may easily become a scaffold for bone ingrowth, and therefore, bone ingrowth in the lattice structure may easily be promoted.

The second region 15 may be located on a lower edge of the top face 11E to surround the top face 11E. In this case, the second region 15 may be located seamlessly on a lower edge of the top face 11E or may be located intermittently on a lower edge of the top face 11E. In the illustrated embodiment, the second region 15 is located seamlessly on a lower edge of the top face 11E to surround the top face 11E.

The second region 15 may be located on an upper edge of the bottom face 11F to surround the bottom face 11F. In this case, the second region 15 may be located seamlessly on an upper edge of the bottom face 11F or may be located intermittently on an upper edge of the bottom face 11F. In the illustrated embodiment, the second region 15 is locate seamlessly on an upper edge of the bottom face 11 F to surround the bottom face 11F.

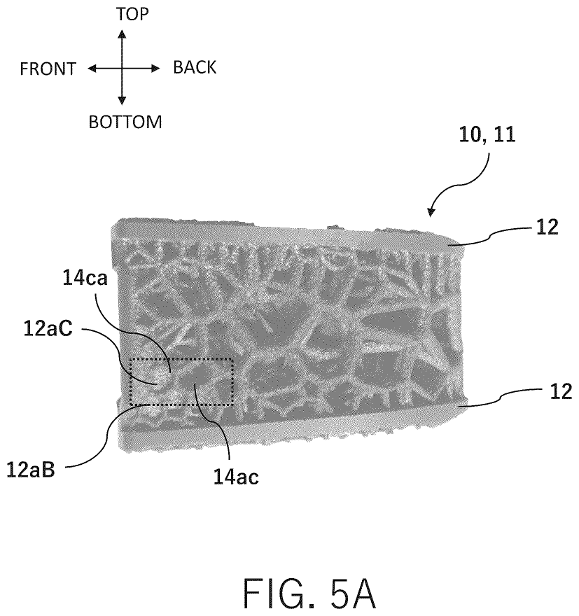

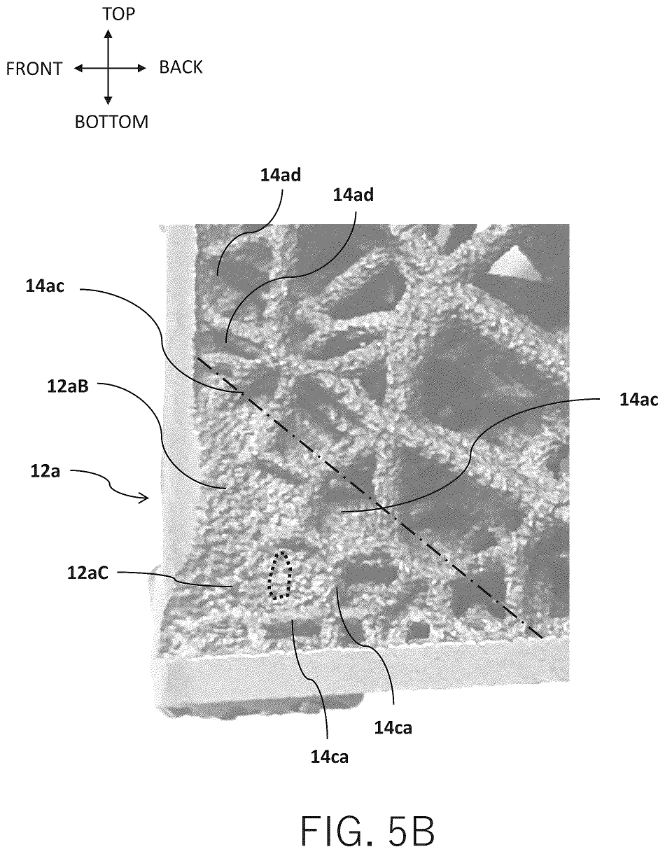

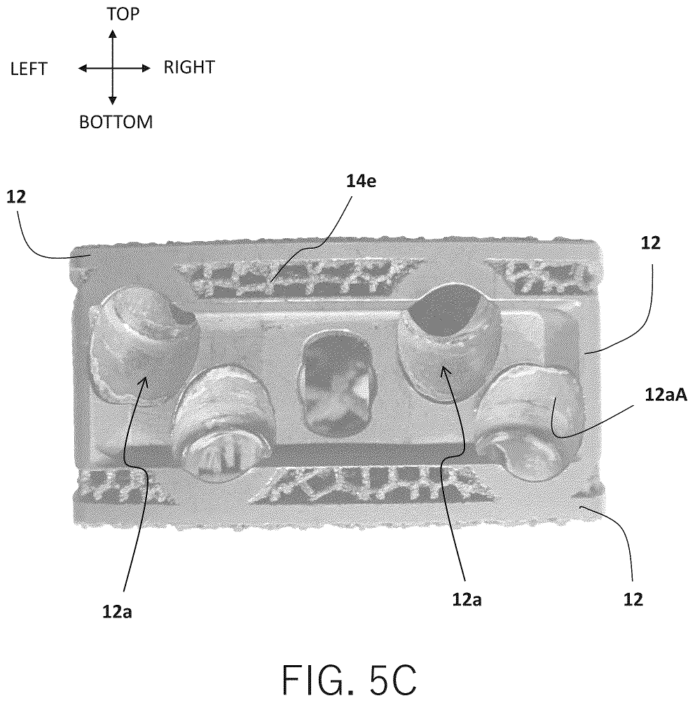

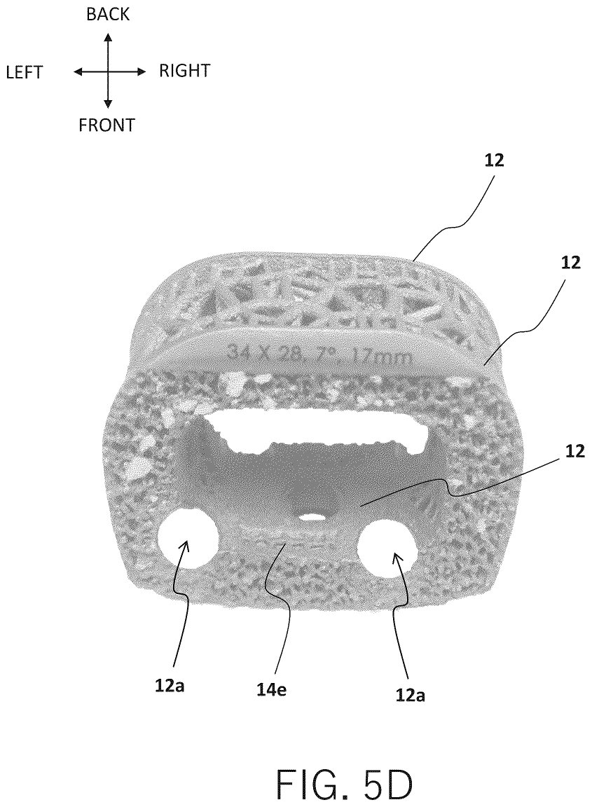

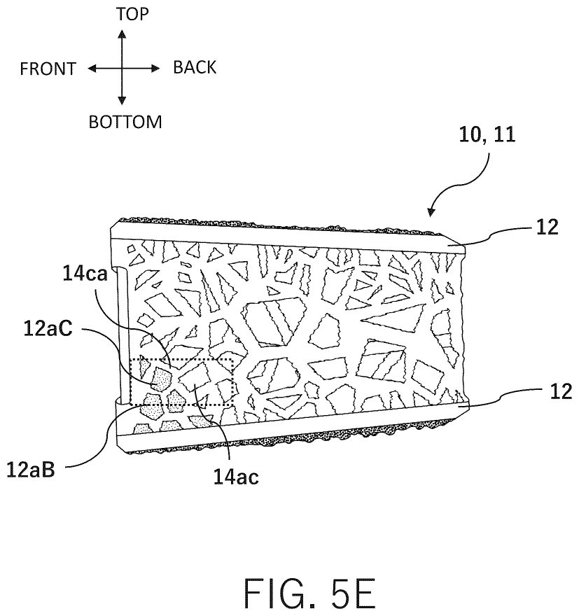

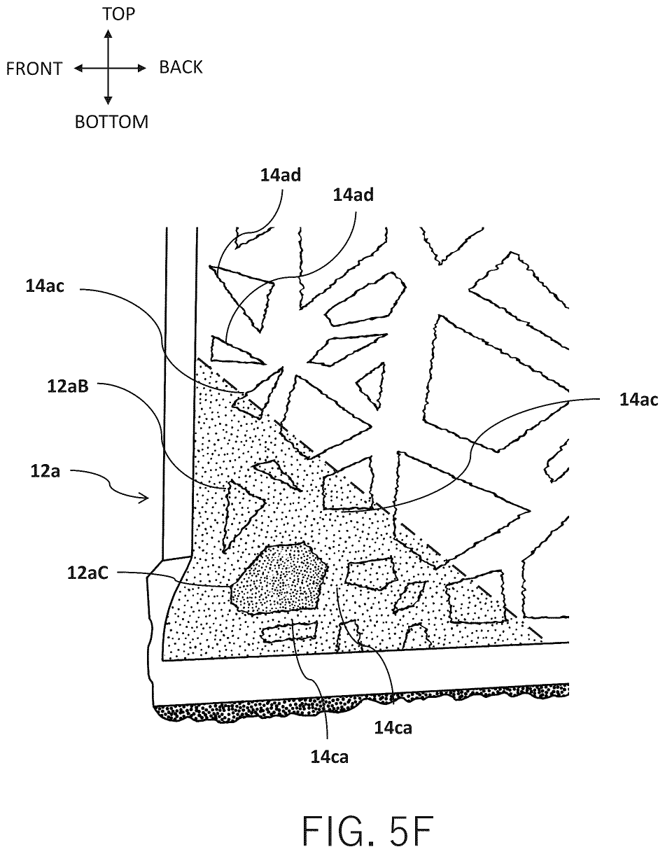

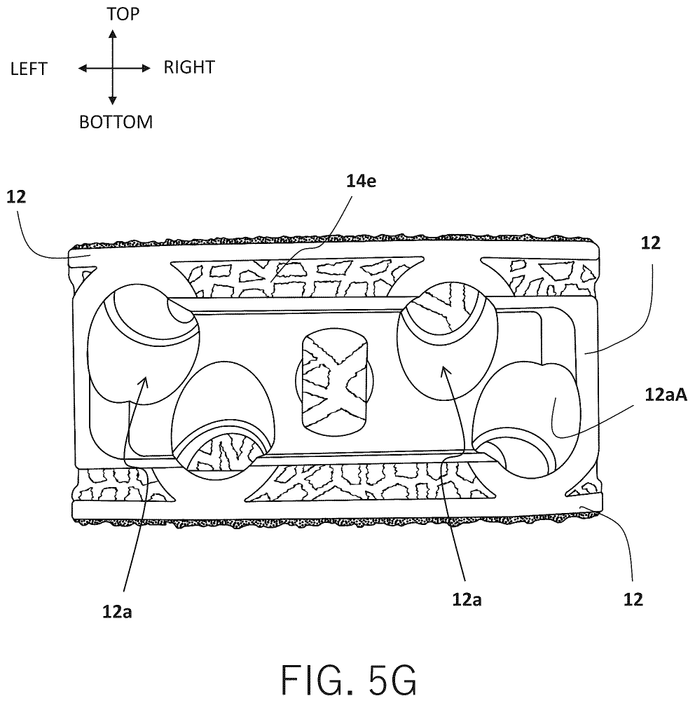

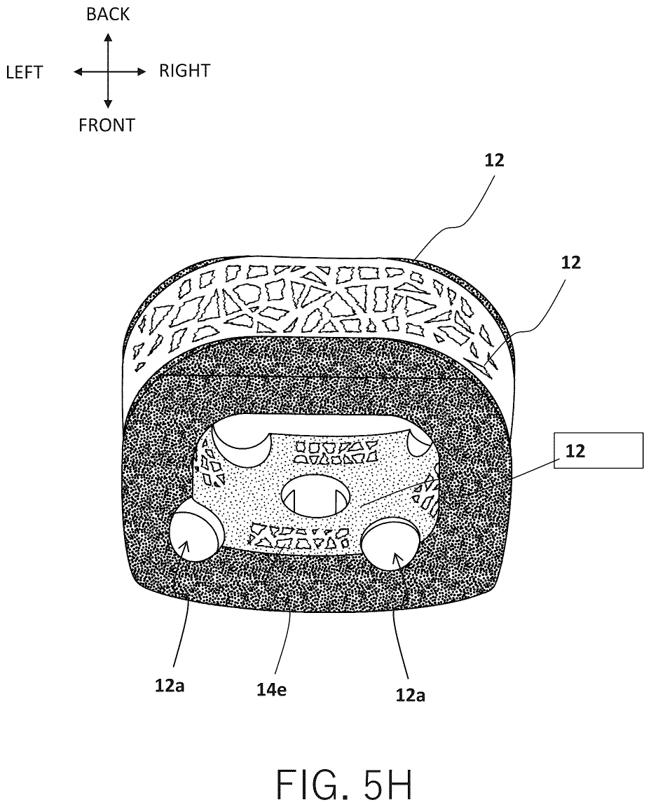

As shown in FIGS. 5A, 5B, 5C, 5D, 5E, 5F, 5G and 5H, a connecting strut 14ca may connect to the outer surface 12aB of the hole 12a. The implant device 10 shown in FIGS. 5A, 5B, 5C, 5D, 5E, 5F, 5G and 5H is a redesigned version of configurations of the implant device 10 shown in FIGS. 4A, 4B, 4C, 4D, 4E and 4F. The dash-dot lines shown in FIGS. 5B and 5F are virtual straight lines that marks the boundary line between the outer surface 12aB of the hole 12a and the connecting struts 14ca. The connecting struts 14ca may connect to the outer surface 12aB of the hole 12a in perpendicular direction to the length direction of the connecting struts 14c. The connecting struts 14ca connected to the hole 12aB may have the same or different features as the other struts 14c, for example. For example, the cross-sectional area in the longitudinal direction of the connecting struts 14ca connected to the hole 12aB may smaller than the cross-sectional area in the longitudinal direction of the other struts 14c.

More specifically, the connecting struts 14ca are configured integrally (to connect) with the outer surface 12aB. Heights (direction perpendicular to the length) from the outside surface 12aB of the connecting struts 14ca, which are configured integrally with the outer surface 12aB, are smaller than a width of the cross-sectional area of the connecting struts 14ca. This can further promote bone ingrowth on the outer surface 12aB. One or more connecting struts 14ca may be included. If there is more than one connecting strut 14ca, the recess 12aC may be configured by the outer surface 12aB and the multiple connecting struts 14ca. Multiple connecting struts 14ca constituting the recess 12aC are shown within the virtual dotted lines in FIGS. 5B and 5F. The depth of the recess 12aC may be, for example, smaller than or equal to the thickness of the struts 14c. On the other hand, the surface roughness of the outer surface 12aB of the hole 12a may be smaller than, for example, the depth of the recess 12aC.

As shown in FIGS. 4, 5A, 5B, 5E and 5F the hole 12a may include a void 14ac surrounded by the outer surface 12aB and the struts 14c. The void 14ac may be, for example, as large as the pores 14a or smaller than the pores 41a and/or 14b. The pores 14ac may be, for example, as large as the pores 14a or smaller than the pores 14a and/or 14b. This may further promote bone ingrowth on the outer surface 12aB.

Alternatively, the pores 14ad surrounded by the front solid surface 12 and the struts 14e may similarly be as large as pores 14a or smaller than pores 14a and/or pores 14b. The pores 14ac and/or the pores 14ad may be smaller than, for example, the pores 14b. This may further promote bone ingrowth on the solid surfaces 12 of the front side.

As shown in FIGS. 3A and 3B, the surfaces of the plurality of struts of the lattice volume 14 may include a third region 171 with a smaller surface roughness compared to the surface roughness of the plurality of struts that is calculated from the measurement results of the entire part of the plurality of the struts. As for the measurement results of the entire part of the plurality of the struts, the measurement of all of the struts is not necessary, but the measurement of representative struts may be sufficient. The third region 171 may be located on the struts 14c. The surface of the struts 14c may further include a fourth region 172 with a larger surface roughness compared to the third region 171. In this case, the third region 171 may be located between the solid surface 12 and the forth region 172. In other words, the solid surface 12, the third region 171, and the forth region 172 may be arranged in that order.

The followings is a method of manufacturing the implant device 10 of the present disclosure. Again, the implant device 10 of the present disclosure is developed using a CAD model and manufactured from a metallic (e.g., titanium) or polymeric (e.g., PEEK) material using an additive manufacturing process, such as 3D printing, or a more traditional manufacturing process. In the additive manufacturing case, the solid surfaces 12 and the lattice volume 14, including the struts 14c, 14d and 14e are integrally formed.

As is described in greater detail herein below, one or more of the solid surfaces 12 may include a porous surface 20 disposed thereon, opposite the lattice volume 14. This porous surface 20 may consist of a simple roughened or patterned surface that promotes bony purchase, or it may consist of a needle-populated secondary lattice volume 22 that further promotes bony purchase. Again, in the additive manufacturing case, the one or more of the solid surfaces 12 and the porous surface(s) 20, including the needle-populated secondary lattice volume(s) 22, are integrally formed. Thus, the implant device 10 includes the solid surfaces 12, the intervening lattice volume 14, and the bone contacting porous surface(s) 20.

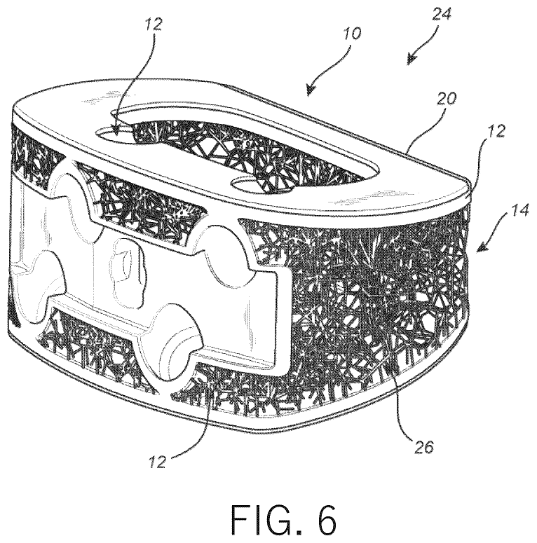

FIG. 6 is schematic diagram illustrating the overall methodology of generating the implant device 10 of FIG. 2. A CAD model 24 is provided that highlights three distinct regions: (1) the solid surfaces 12, (2) the lattice volume 14, and (3) the porous surface(s) 20. Using a software application, a Voronoi Volume Lattice (VVL) 26 is generated in the lattice volume 14, which replaces the solid material that would otherwise be present within the lattice volume 14. Generation of this VVL 26 requires the selection of random and/or ordered points within the lattice volume 14, which become the centers of the voids or pores of the VVL 26. Here, the ordered points may be manually generated, generated based on a solid geometry, and/or generated by a mathematical equation. To achieve the desired void or pore density variation, the lattice volume 14 may be segmented into sub-volumes or regions with VVL granularity that is more fine in a sub-volume adjacent to a solid surface 12 and more coarse in a subvolume remote from a solid surface 12, for example, provided the VVL 26 is interconnected at the sub-volume interfaces. Preferably, two discrete sets of points are selected and utilized to generate the VVL 26.

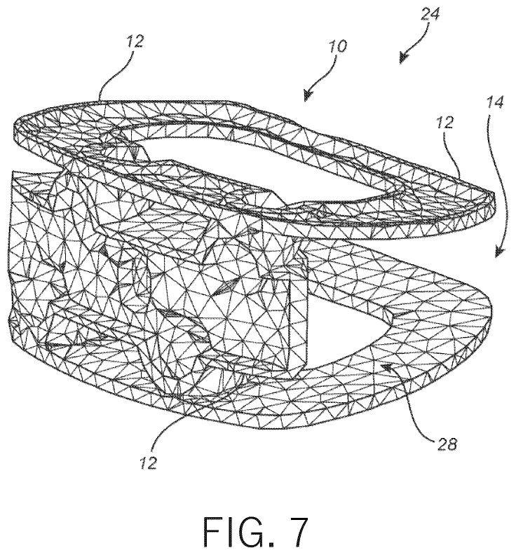

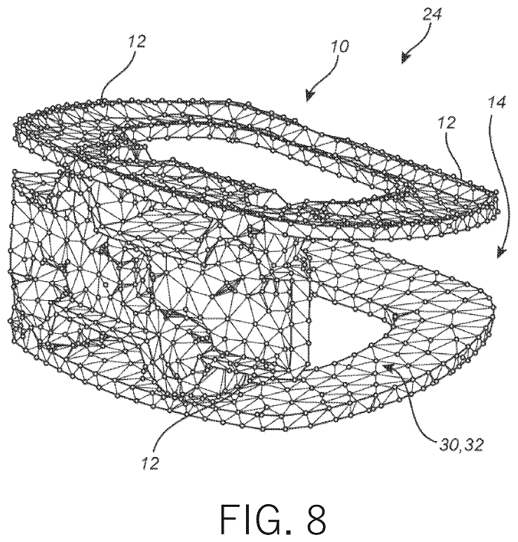

Referring now specifically to FIG. 7, the first discrete set of points 32 (FIG. 8) is selected by applying a relatively fine virtual mesh 28 to the lattice volume 14. This virtual mesh 28 may be uniform and utilizes edge lengths on the order of 1.75-2 mm, for example, with the virtual mesh intersecting the solid surfaces 12. It will be readily apparent to those of ordinary skill in the art that other dimensions may be used equally.

Referring now specifically to FIG. 8, the vertices 30 of the virtual mesh 28 (FIG. 7) with the solid surface 12 are then selected as the first discrete set of points 32.

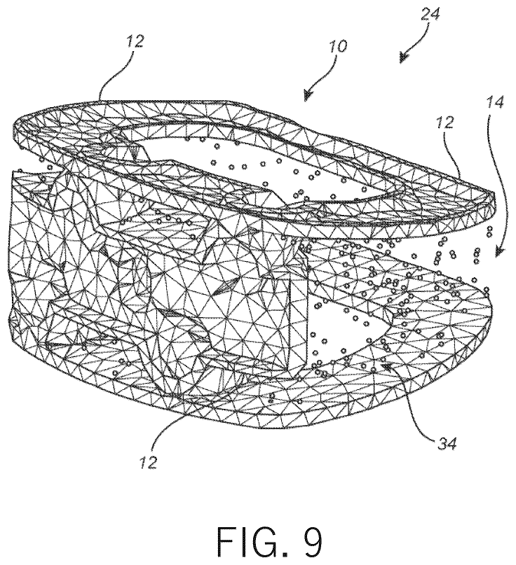

Referring now specifically to FIG. 9, the second discrete set of points 34 is selected or generated at random within the lattice volume 14, and are spaced by about 3mm adjacent to the solid surfaces 12 and by about 4 mm remote from the solid surfaces 12. It will be readily apparent to those of ordinary skill in the art that other dimensions may be used equally.

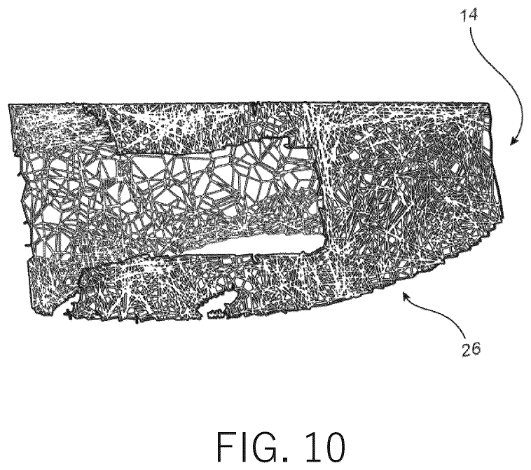

Referring now specifically to FIG. 10, the first discrete set of points 32 (FIG. 8) and the second discrete set of points 34 (FIG. 9) are combined and used to generate the VVL 26, with the points 32, 34 representing the centers of the voids or pores (or, alternatively, the intersections of the struts of the VVL 26). The VVL 26 is then trimmed to fit the lattice volume 14. The result is a skeleton of the lattice volume 14. With all struts having the same cross-sectional diameter, the lattice volume 14 is thicker and less porous adjacent to the solid surfaces 12 (FIGS. 2-9) and finer and more porous remote from the solid surfaces 12. Because more points 32 and voids or pores are provided adjacent to the solid surfaces 12.

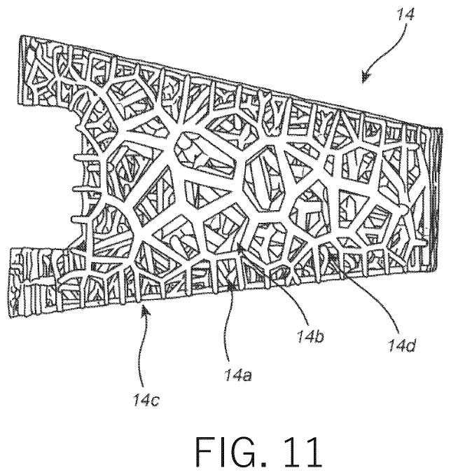

Referring now specifically to FIG. 11, the skeleton of the lattice volume 14 is thickened. The struts 14c adjacent to the solid surfaces 12 (FIGS. 2-9) are increased to about 0.45 mm, for example, while the struts 14d remote from the solid surfaces 12 are increased to about 1 mm, for example. It will be readily apparent to those of ordinary skill in the art that other dimensions may be used. Further, the thickness of a given strut 14c, 14d and 14e may be varied along its length. Because the smaller struts 14c adjacent to the solid surfaces 12 surround smaller, more dense pores 14a, and the larger struts 14d remote from the solid surfaces 12 surround larger, less dense pores 14b, the two regions are comparable in a percent porosity, which may be 60-85%, for example. It will be readily apparent to those of ordinary skill in the art that other porosities may be used equally. The struts 14c, 14d and 14e may have any suitable shape with a cross-sectional shape cut in a direction perpendicular to the length direction. The cross-sectional shapes of the struts 14c, 14d and 14e may be, for example, circular, oval, triangular, square, rectangular, pentagonal, octagonal, irregular and their combined shapes.

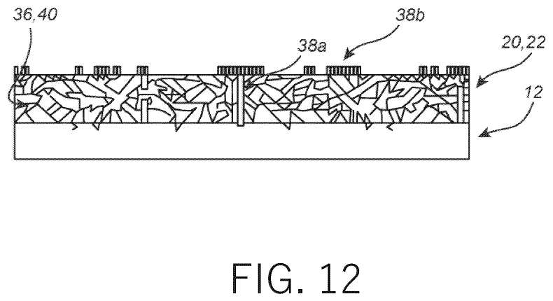

FIG. 12 is a schematic diagram illustrating one illustrative embodiment of the needle-populated porous structure 22 that may cover the exterior of the implant device 10 (FIGS. 2-9). The implant device 10 may include at least one of the following: a primary structure 12; and at least one secondary lattice or porous surface portion 36 formed on at least one exterior portion of the primary structure 12, the at least one surface portion 36 located such that it engages with a patient's bone when the implant 10 is implanted in a patient. Such needle-populated, metallic surface portion 22 may contain, for example, a collection of at least fifty, a hundred, two hundred, five-hundred or more needles 38a, 38b, and may be further characterized by at least one, two, three, four, five or more of the following characteristics:

(a) the needles 38a, 38b in the collection are all oriented substantially normal to the surface portion 36; (b) the needles 38a, 38b in the collection are all oriented in substantially the same direction, with the direction being other than normal to the surface portion 36; (c) the needles 38a, 38b in the collection are all oriented in substantially the same direction, with the direction being other than normal to the surface portion 36, but within 15 degrees from the normal direction; (d) the needles 38a, 38b in the collection are all oriented in substantially the same direction, with the direction being other than normal to the surface portion 36, and more than 15 degrees from the normal direction; (e) the collection includes needles 38a, 38b oriented in at least three different directions relative to the surface portion 36; (l) the collection includes needles 38a, 38b oriented in at least five different directions relative to the surface portion 36, with all of the needles oriented within 20 degrees from the surface portion normal direction; (g) all of the needles 38a, 38b in the collection have substantially the same height; (h) the collection includes needles 38a, 38b of at least three different heights;

(i) all of the needles 38a, 38b in the collection have substantially the same shape; (j) the collection includes needles 38a, 38b of at least two different shapes; (k) the needles 38a, 38b are distributed substantially uniformly over the surface portion 36; (l) the needles 38a, 38b are distributed non-uniformly over the surface portion 36; (m) all of the needles 38a in the collection are anchored to the primary structure 12; (n) most of the needles 38a in the collection are anchored to the primary structure 12; (o) most of the needles 38b in the collection are anchored to structural elements contained within an osteo-porous, osteo-derived or trabecular coating 36 on the at least one exterior portion of the primary structure 12; and/or (p) all of the needles 38b in the collection are anchored to structural elements contained within an osteo-porous, osteo-derived or trabecular coating 36 on the at least one exterior portion of the primary structure 12. The at least one exterior portion 22 preferably includes at least one osteo-porous surface 36, which may comprise at least one osteo-derived surface 36. The at least one osteo-porous surface 36 and the needles 38a, 38b may be simultaneously formed by an additive manufacturing process.

The exemplary manufacturing flow starts with a spongy bone sample, which is micro-scanned to obtain 3D scan data, which is then processed into solid model data representing an osteo-porous or osteo-derived texture. This texture data is then combined with data representing the overall implant geometry to create a fabrication file for use by any of the manufacturing steps that follow. The fabrication file may utilize any recognizable solid model specification, such as “amf” format or “stl” format, and may be embodied on any sort of permanent, non-transitory storage medium (e.g., CD, CD-ROM, flash), semi-permanent (e.g., SRAM) or transitory (e.g., DRAM) storage medium, or embodied in a coded data signal.

An additional step is taken that adds outwardly -protruding “ needles” 38a, 38b on the outer surface(s) of the osteo-porous and/or osteo-derived coating(s) 36. Such needles 38a, 38b substantially increase the coefficient of friction of the implant surface 22. Having a high coefficient of friction is clinically advantageous because it provides stronger initial fixation, which is important before bone is able to grow onto/into the porous structure 20. Such needles 38a, 38b can be uniformly or non-uniformly distributed along the porous surface. Likewise, various shapes for the needles 38a, 38b are possible, including rectangular, pyramidal, conical, tube-shaped, barbed, etc. Also, the needles 38a, 38b need not be oriented exactly normal to the exterior surface, but are preferably oriented in a substantially normal (e.g., within +/−15 degrees from normal) orientation. Furthermore, the orientation and/or shape of all needles 38a, 38b need not be the same, and the needles 38a, 38b may be rendered on selected portions, or the entirety, of the exterior coated surface(s) 20.

The methodology generates and provides a surface 22 that includes the implant body 12 and a porous layer 36 that is disposed directly adjacent to the implant body 12. The porous layer 36 can be additively manufactured on top of the implant body 12, or can be additively manufactured with the implant body 12. The porous layer 36 consists of a bone-interfacing lattice 40 of macroscopic, randomly distributed stochastic struts of various thicknesses, shapes, and intersection points. This lattice 40 is comparable to cancellous bone in terms of pore size and overall porosity, thus it elicits a favorable bone response when applied to the bone-opposition surfaces of the associated implant 10 to which it is applied. The needles 38a, 38b are additively manufactured with the porous layer 36 and/or the implant body 12 and some or all of the needles 38a protrude from and are anchored directly to the implant body 12, through the porous layer 36, and from the bone-opposition surface of the porous layer 36, forming regularly or randomly-arranged friction structures protruding from the bone-opposition surface of the porous layer 36. This provides advantageous needle strength and stability. Within the porous layer 36, these penetrating needles 38a are integrally formed with or otherwise anchored to adjacent of the struts of the lattice 40, again providing advantageous needle strength and stability. In one preferred embodiment, all needles 38a are planted 0.004-0.006 in. into the solid substrate, either physically or for behavioral modeling purposes (having a corresponding support stiffness), and extend about 0.008 in. above the bone-opposition surface of the porous layer 36, with a plurality of intervening lattice strut connections along the length of each needle 38a. Here, the needles 38a are 0.2 mm×0.2 mm rectangular prisms with constant cross-sections, for example. The preferred needle density is 0.3 needles/mm² or 1 needle 38a every 3.33 mm² for optimal bone friction engagement. The needles 38a are largely disposed normal to the bone-opposition surface of the porous layer 36 and the implant body 12, but may be angled with respect to one another due to curvature of the bone-opposition surface of the porous layer 36 and the implant body 12. The finished porous layerneedle construct is blasted with calcium phosphate or otherwise surface treated to promote roughness of the resulting bone-engagement structure. Alternatively, an osteo-porous, osteo-derived, and/or trabecular coating 36 with needles 38b anchored only to the bone-opposition surface of the osteo-porous, osteo-derived, and/or trabecular coating 36 and not the underlying implant body 12 may be utilized.

Slightly irregular secondary lattices 40 are ideally adapted for additive manufacturing in accordance with the present disclosure. Node perturbation refers to the location of intersecting struts. Such intersection locations can be randomized such that the node deviates from a uniform lattice by a randomized distance or degree. Strut size randomization refers to a deviation in cross-sectional dimension (e.g., strut diameter), as well as shape and length. Discrete struts in a lattice could have different cross-sectional sizes, or the struts could have a diameter gradient from one end to the other. These parameters can be randomized for greater diversity in the lattice's geometry. Such slightly-irregular lattices can be used to fabricate any sort of medical implant for which regular lattices might otherwise be used.

It should be understood that the novel structures disclosed and enabled by the present disclosure are not limited exclusively to those manufactured using additive manufacturing. Indeed, as persons skilled in the art will appreciate, other known surface modification techniques may be used to produce the osteoporous, osteo- derived, and/or needle-containing textures of the inventive implants.

The methodology of the present disclosure provides a surface that includes the implant body (or “melt”) and a porous layer (or “structure”) that is disposed directly adjacent to the implant body. The porous layer can be additively manufactured on top of the implant body, or can be additively manufactured with the implant body. The porous layer consists of a bone-interfacing lattice of macroscopic, randomly distributed stochastic struts of various thicknesses, shapes, and intersection points. This lattice is comparable to cancellous bone in terms of pore size and overall porosity, thus it elicits a favorable bone response when applied to the bone-opposition surfaces of the associated implant to which it is applied. The generation of the overall structure is accomplished through several CAD modeling programs in the following data preparation process flow. CAD modeling software is used to generate the design envelope (i.e. the volumes) and spatial relationship (i.e. the overlap) of the various structural elements. The overall structure is comprised of three specific volume elements: the melt volume, the structure volume, and the needle volume. The melt volume is the CAD volume that defines the solid substrate that the structure and needles interface and overlap to ensure a mechanical interface during the additive manufacturing process. For most scenarios, the melt volume is the bulk of the device. The structure volume is the CAD volume that defines the virtual boundary conditions for which the random, stochastic structure will be generated. The structure volume is purely solid and without any lattice. The needle volume is the CAD volume that defines the virtual boundary conditions for which the random, protrusions will extend beyond the regions of the structure volume. The needle volume is purely solid and without any needles (i.e. protrusions). A CAD assembly combines the melt volume, structure volume, and needle volume part models. The structure volume and needle volume elements overlap with the melt volume within the defined coordinate system through the mate interface GUI. This overlap is based on the resolution and accuracy of the intended additive manufacturing technology for which the device will be manufactured. After the CAD assembly has been defined, the models are exported as a “.stl” file format. The files are imported into additional CAD software and used to generate the structure and needles from the structure volume and needle volume elements that were previously defined. Using the structure volume, the user executes the graphical user interface (GUI) algorithm. This algorithm applies a unit cell within a defined volume relative to the CAD environment's coordinate system. The algorithm executes a Boolean operation between the array of unit cells and the structure-volume to yield only the portions of the unit cell within the volume. The overall structure utilizes a porous structure unit cell of defined dimensions, shape, and volume. The algorithm is used to duplicate the porous structure unit cell as an array across the structure volume element and then trim the unit cells within the boundaries of the structure volume. The result is the structure; a random, stochastic lattice that fills the volume of the original structure volume envelope. Similar to structure generation, needles are generated via an intersection Boolean operation between the needle volume element and a preprogrammed file that is generated by an equation-driven algorithm. The preprogrammed needle-element is imported into the CAD software and spatially-aligned with the needle volume. The Boolean is executed and the resulting geometry is an array of randomly located needles (i.e. protrusions) within the boundaries previously defined by the needle volume. After successfully generating the structure and needles, the components are exported as “.stl” files. The files are then imported into additive manufacturing technology-specific software programs in preparation for the additive manufacturing process. The technology-specific software programs slice the CAD models at a defined thickness acceptable for the additive manufacturing equipment, define the sequence of part build order, and apply exposure strategies. The result of these programs is a build file that is imported and executed on the additive manufacturing machine to yield a physical part.

It is to be recognized that, depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

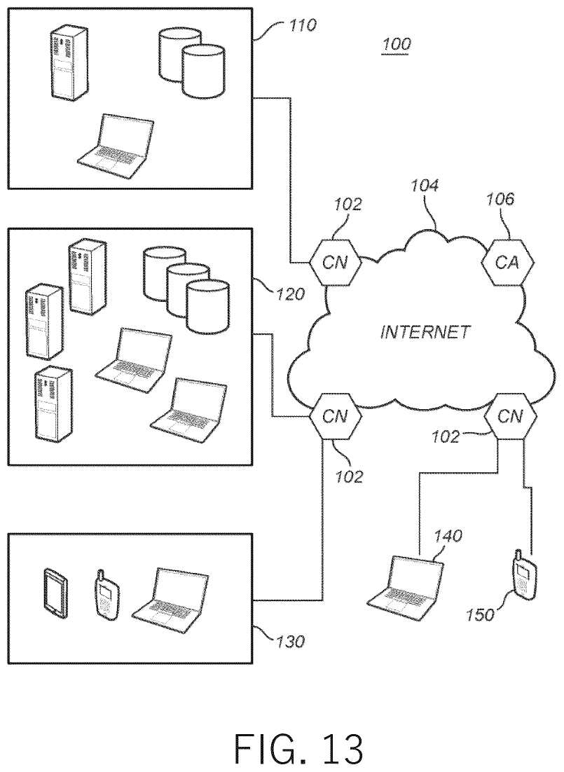

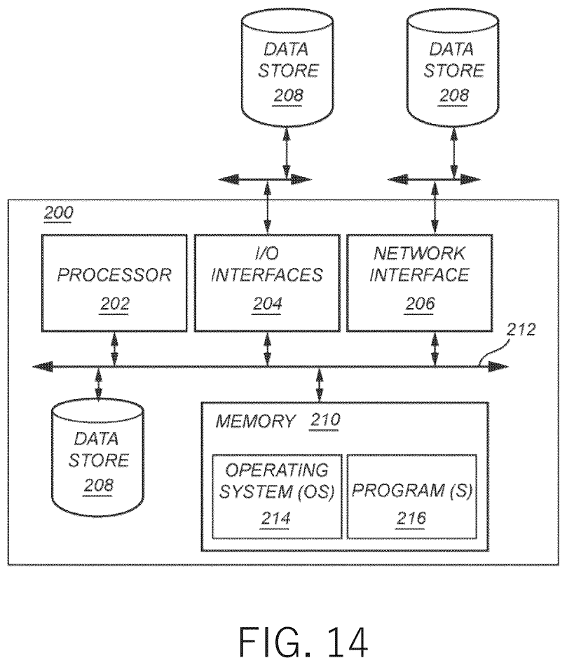

FIG. 13 is a network diagram of a cloud-based system 100 for implementing various cloud-based services of the present disclosure. The cloud-based system 100 includes one or more cloud nodes (CNs) 102 communicatively coupled to the Internet 104 or the like. The cloud nodes 102 may be implemented as a server 200 (as illustrated in FIG. 14) or the like and can be geographically diverse from one another, such as located at various data centers around the country or globe. Further, the cloud-based system 100 can include one or more central authority (CA) nodes 106, which similarly can be implemented as the server 200 and be connected to the CNs 102. For illustration purposes, the cloud-based system 100 can connect to a regional office 110, headquarters 120, various employee' s homes 130, laptops/desktops 140, and mobile devices 150, each of which can be communicatively coupled to one of the CNs 102. These locations 110, 120, and 130, and devices 140 and 150 are shown for illustrative purposes, and those skilled in the art will recognize there are various access scenarios to the cloud-based system 100, all of which are contemplated herein. The devices 140 and 150 can be so-called road warriors, i.e., users off-site, on-the-road, etc. The cloud-based system 100 can be a private cloud, a public cloud, a combination of a private cloud and a public cloud (hybrid cloud), or the like.

Again, the cloud-based system 100 can provide any functionality through services such as software-as-a-service (SaaS), platform-as-a-service, infrastructure-as-a-service, security-as-a-service, Virtual Network Functions (VNFs) in a Network Functions Virtualization (NFV) Infrastructure (NFVI), etc. to the locations 110, 120, and 130 and devices 140 and 150. Previously, the Information Technology (IT) deployment model included enterprise resources and applications stored within an enterprise network (i.e., physical devices), behind a firewall, accessible by employees on site or remote via Virtual Private Networks (VPNs), etc. The cloud-based system 100 is replacing the conventional deployment model. The cloud-based system 100 can be used to implement these services in the cloud without requiring the physical devices and management thereof by enterprise IT administrators.

Cloud computing systems and methods abstract away physical servers, storage, networking, etc., and instead offer these as on-demand and elastic resources. The National

Institute of Standards and Technology (NIST) provides a concise and specific definition which states cloud computing is a model for enabling convenient, on-demand network access to a shared pool of configurable computing resources (e.g., networks, servers, storage, applications, and services) that can be rapidly provisioned and released with minimal management effort or service provider interaction. Cloud computing differs from the classic client-server model by providing applications from a server that are executed and managed by a client's web browser or the like, with no installed client version of an application necessarily required. Centralization gives cloud service providers complete control over the versions of the browser-based and other applications provided to clients, which removes the need for version upgrades or license management on individual client computing devices. The phrase “software as a service” (SaaS) is sometimes used to describe application programs offered through cloud computing. A common shorthand for a provided cloud computing service (or even an aggregation of all existing cloud services) is “the cloud.” The cloud-based system 100 is illustrated herein as one example embodiment of a cloud-based system, and those of ordinary skill in the art will recognize the systems and methods described herein are not necessarily limited thereby.

FIG. 14 is a block diagram of a server 200, which may be used in the cloudbased system 100 (FIG. 13), in other systems, or standalone. For example, the CNs 102 (FIG. 13) and the central authority nodes 106 (FIG. 13) may be formed as one or more of the servers 200. The server 200 may be a digital computer that, in terms of hardware architecture, generally includes a processor 202, input/output (I/O) interfaces 204, a network interface 206, a data store 208, and memory 210. It should be appreciated by those of ordinary skill in the art that FIG. 14 depicts the server 200 in an oversimplified manner, and a practical embodiment may include additional components and suitably configured processing logic to support known or conventional operating features that are not described in detail herein. The components (202, 204, 206, 208, and 210) are communicatively coupled via a local interface 212. The local interface 212 may be, for example, but is not limited to, one or more buses or other wired or wireless connections, as is known in the art. The local interface 212 may have additional elements, which are omitted for simplicity, such as controllers, buffers (caches), drivers, repeaters, and receivers, among many others, to enable communications. Further, the local interface 212 may include address, control, and/or data connections to enable appropriate communications among the aforementioned components.

The processor 202 is a hardware device for executing software instructions. The processor 202 may be any custom made or commercially available processor, a central processing unit (CPU), an auxiliary processor among several processors associated with the server 200, a semiconductor-based microprocessor (in the form of a microchip or chipset), or generally any device for executing software instructions. When the server 200 is in operation, the processor 202 is configured to execute software stored within the memory 210, to communicate data to and from the memory 210, and to generally control operations of the server 200 pursuant to the software instructions. The I/O interfaces 204 may be used to receive user input from and/or for providing system output to one or more devices or components.

The network interface 206 may be used to enable the server 200 to communicate on a network, such as the Internet 104 (FIG. 13). The network interface 206 may include, for example, an Ethernet card or adapter (e.g., lOBaseT, Fast Ethernet, Gigabit Ethernet, or lOGbE) or a Wireless Local Area Network (WLAN) card or adapter (e.g., 802.11 a/b/g/n/ac). The network interface 206 may include address, control, and/or data connections to enable appropriate communications on the network. A data store 208 may be used to store data. The data store 208 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 208 may incorporate electronic, magnetic, optical, and/or other types of storage media. In one example, the data store 208 may be located internal to the server 200, such as, for example, an internal hard drive connected to the local interface 212 in the server 200. Additionally, in another embodiment, the data store 208 may be located external to the server 200 such as, for example, an external hard drive connected to the I/O interfaces 204 (e.g., a SCSI or USB connection). In a further embodiment, the data store 208 may be connected to the server 200 through a network, such as, for example, a network-attached file server.

The memory 210 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 210 may incorporate electronic, magnetic, optical, and/or other types of storage media. Note that the memory 210 may have a distributed architecture, where various components are situated remotely from one another but can be accessed by the processor 202. The software in memory 210 may include one or more software programs, each of which includes an ordered listing of executable instructions for implementing logical functions. The software in the memory 210 includes a suitable operating system (O/S) 514 and one or more programs 216. The operating system 214 essentially controls the execution of other computer programs, such as the one or more programs 216, and provides scheduling, input-output control, file and data management, memory management, and communication control and related services. The one or more programs 216 may be configured to implement the various processes, algorithms, methods, techniques, etc. described herein.

It will be appreciated that some embodiments described herein may include one or more generic or specialized processors (“one or more processors”) such as microprocessors; central processing units (CPUs); digital signal processors (DSPs); customized processors such as network processors (NPs) or network processing units (NPUs), graphics processing units (GPUs), or the like; field programmable gate arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more applicationspecific integrated circuits (ASICs), in which each function or some combinations of certain of the functions are implemented as custom logic or circuitry. Of course, a combination of the aforementioned approaches may be used. For some of the embodiments described herein, a corresponding device in hardware and optionally with software, firmware, and a combination thereof can be referred to as “circuitry configured or adapted to,” “logic configured or adapted to,” etc. perform a set of operations, steps, methods, processes, algorithms, functions, techniques, etc. on digital and/or analog signals as described herein for the various embodiments.

Moreover, some embodiments may include a non-transitory computer-readable storage medium having computer-readable code stored thereon for programming a computer, server, appliance, device, processor, circuit, etc. each of which may include a processor to perform functions as described and claimed herein. Examples of such computer-readable storage mediums include, but are not limited to, a hard disk, an optical storage device, a magnetic storage device, a Read-Only Memory (ROM), a Programmable Read-Only Memory (PROM), an Erasable Programmable Read-Only Memory (EPROM), an Electrically Erasable Programmable Read-Only Memory (EEPROM), flash memory, and the like. When stored in the non-transitory computer- readable medium, software can include instructions executable by a processor or device (e.g., any type of programmable circuitry or logic) that, in response to such execution, cause a processor or the device to perform a set of operations, steps, methods, processes, algorithms, functions, techniques, etc. as described herein for the various embodiments.

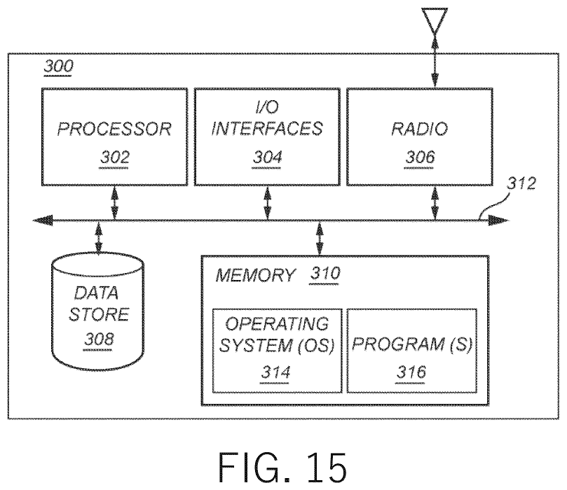

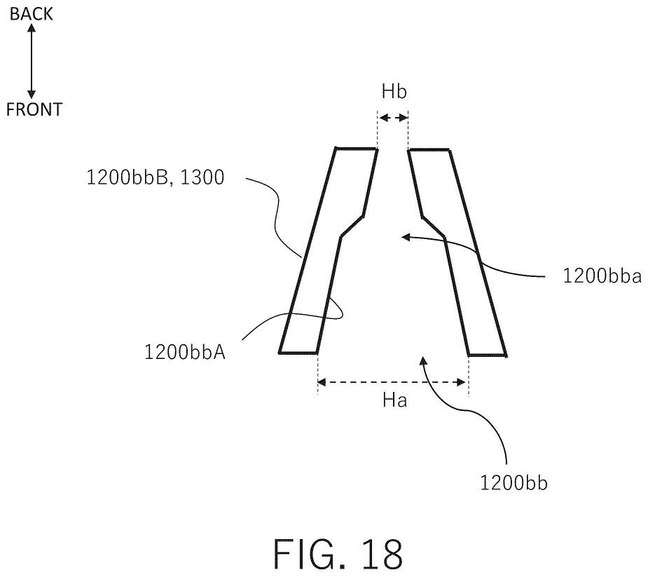

FIG. 15 is a block diagram of a user device 300, which may be used in the cloud-based system 100 (FIG. 13) or the like. Again, the user device 300 can be a smartphone, a tablet, a smartwatch, an Internet of Things (IoT) device, a laptop, a virtual reality (VR) headset, etc. The user device 300 can be a digital device that, in terms of hardware architecture, generally includes a processor 302, I/O interfaces 304, a radio 306, a data store 308, and memory 310. It should be appreciated by those of ordinary skill in the art that FIG. 18 depicts the user device 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 can be, for example, but is not limited to, one or more buses or other wired or wireless connections, as is known in the art. The local interface 312 can have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 can be any custom made or commercially available processor, a CPU, an auxiliary processor among several processors associated with the user device 300, a semiconductor-based microprocessor (in the form of a microchip or chipset), or generally any device for executing software instructions. When the user device 300 is in operation, the processor 302 is configured to execute software stored within the memory 310, to communicate data to and from the memory 310, and to generally control operations of the user device 300 pursuant to the software instructions. In an embodiment, the processor 302 may include a mobile optimized processor such as optimized for power consumption and mobile applications. The I/O interfaces 304 can be used to receive user input from and/or for providing system output. User input can be provided via, for example, a keypad, a touch screen, a scroll ball, a scroll bar, buttons, a barcode scanner, and the like. System output can be provided via a display device such as a liquid crystal display (LCD), touch screen, and the like.

The radio 306 enables wireless communication to an external access device or network. Any number of suitable wireless data communication protocols, techniques, or methodologies can be supported by the radio 306, including any protocols for wireless communication. The data store 308 may be used to store data. The data store 308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media.

Again, the memory 310 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 can include one or more software programs, each of which includes an ordered listing of executable instructions for implementing logical functions. In the example of FIG. 15, the software in the memory 310 includes a suitable operating system 314 and programs 316. The operating system 314 essentially controls the execution of other computer programs and provides scheduling, input-output control, file and data management, memory management, and communication control and related services. The programs 316 may include various applications, add-ons, etc. configured to provide end user functionality with the user device 300. For example, example programs 316 may include, but not limited to, a web browser, social networking applications, streaming media applications, games, mapping and location applications, electronic mail applications, financial applications, and the like. In a typical example, the end-user typically uses one or more of the programs 316 along with a network such as the cloud-based system 100 (FIG. 13).

Although the present disclosure is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following non-limiting claims for all purposes. Although the present disclosure is illustrated and described with the lattice volume 14, the implant device 10 may not include the lattice volume 14.

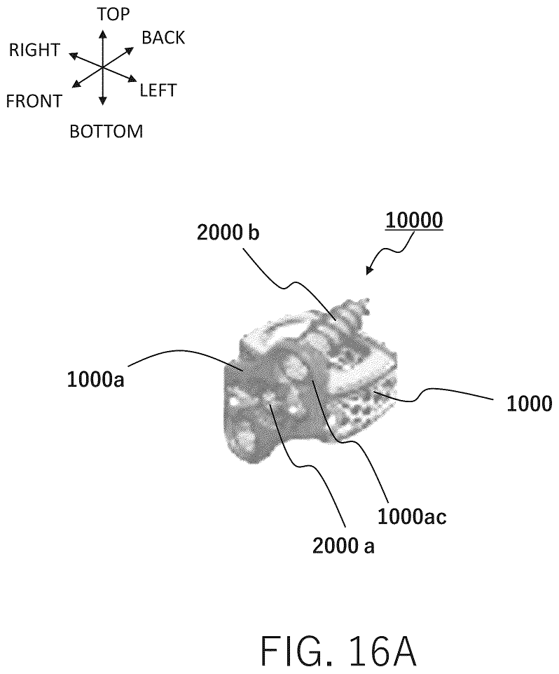

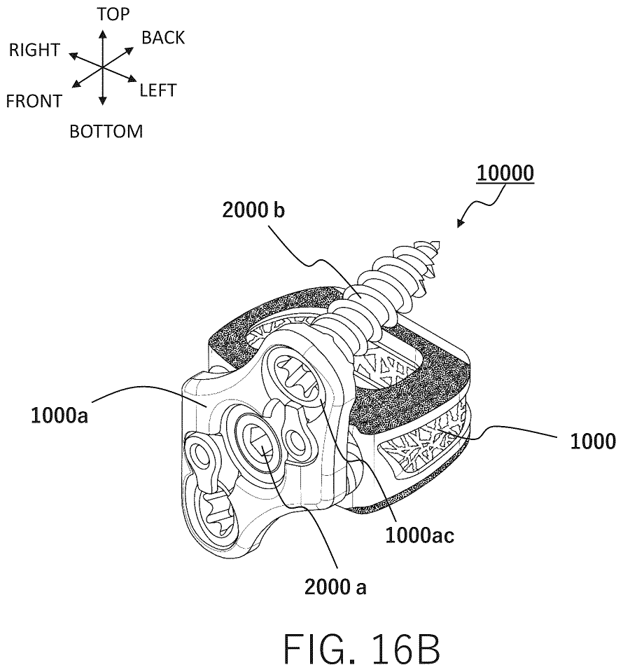

While the above embodiment describes an ALIF cage, it may be used for a cage for a cervical spine. For example, an implant system 10000 for the cervical spine is shown in FIGS. 16A and 16B. The same symbols are given for description of the same configurations as the implant device 10.

The implant system 10000 may be fixed to a cervical vertebrae using, for example, a plate 1000a. The plate 1000a may fixed to an implant device 1000 with screws 2000a, for example. The plate 1000a may include a hole 1000ac through which the screws 2000b fix to a cervical vertebrae.

The implant device 1000 may be configured to be smaller than, for example, an ALIF cage (implant device 10). The implant system 10000 may include the plate 1000a, but may not include the plate 1000a. Specifically, the implant system 10000 has a hole through which a screw 2000b for cervical spine fixation passes, and the implant system 10000 is fixed to the cervical spine through the screw 2000b.

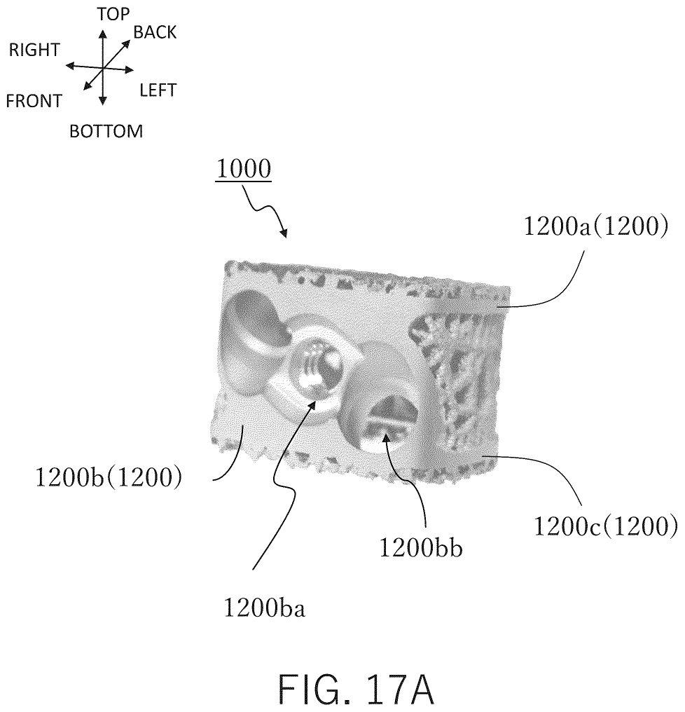

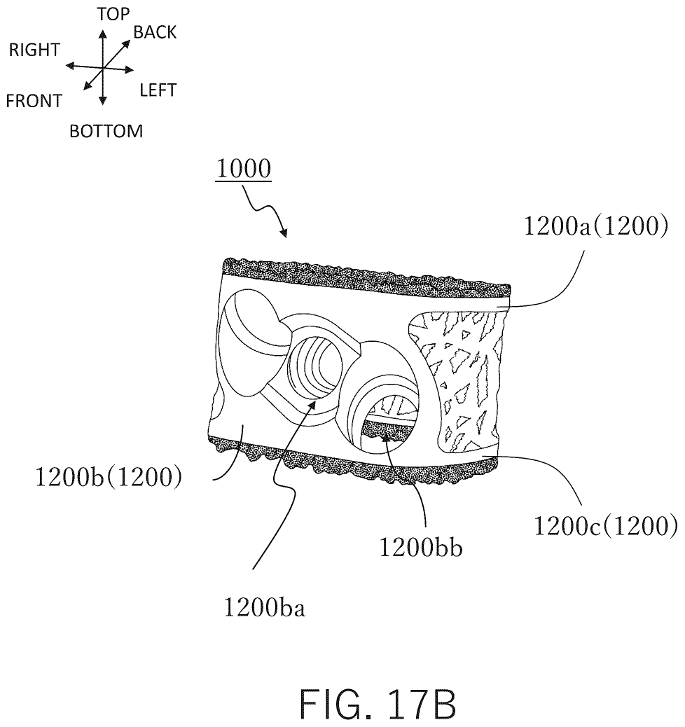

The implant device 1000 is largely composed of a solid surface 1200. More specifically, the implant device 1000 consists of, for example, a solid surface 1200a of the top side, a solid surface 1200b of the front side and a solid surface 1200c of the bottom side, as shown in FIGS. 17A and 17B. The solid surface 1200b of the front side, for example, may not include the struts 14e or may include the struts 14e. The solid surface 1200b of the front side may include, for example, a screw hole 1200ba for securing the plate 1000a and a screw hole 1200bb through which the screw 2000b for cervical spine fixation passes. The screw hole(s) 1200ba are configured to engage the screw(s) 2000a that secure the plate 1000a. The screw hole(s) 1200bb may include a larger section(s) than the screw(s) 2000b so that the screw(s) 2000b can pass through it.

As shown in FIG. 18, a portion of the cross section of the screw hole(s) 1200bb may be smaller toward the back. Width Ha of the front opening of the screw hole(s) 1200bb may be smaller than width Hb of the rear opening of the screw hole 1200bb. Width of the screw holes 1200bb may gradually decrease from front to rear. In addition, the screw holes 1200bb may have, for example, a portion 1200bba that is narrower than the other portions.

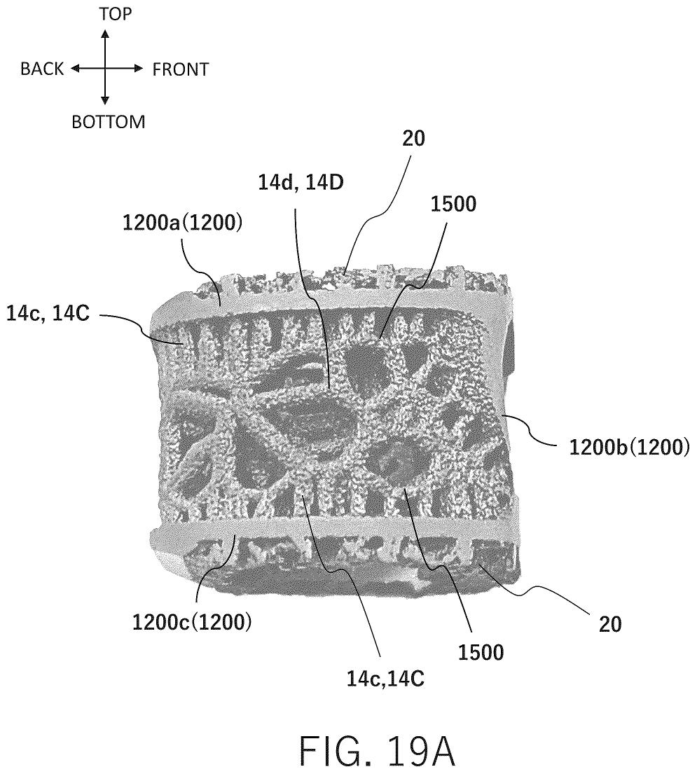

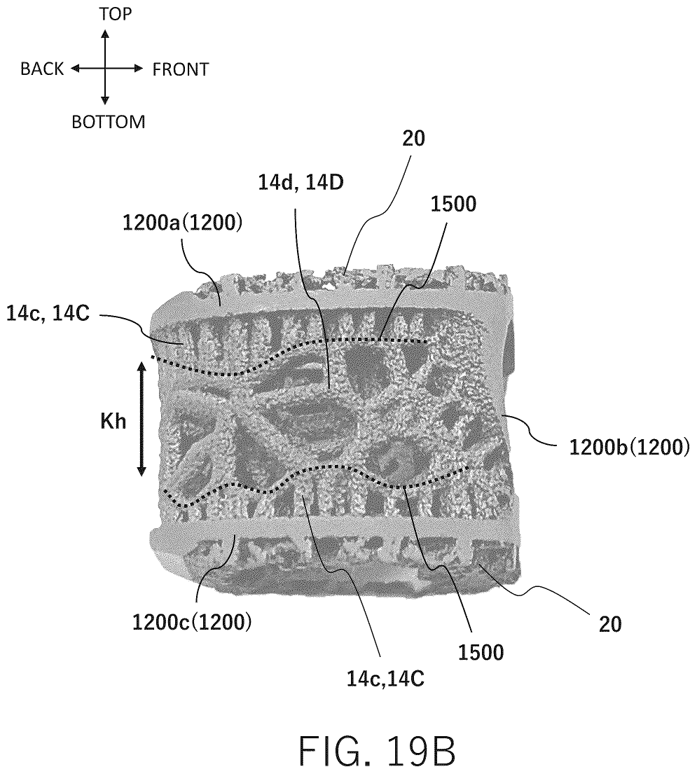

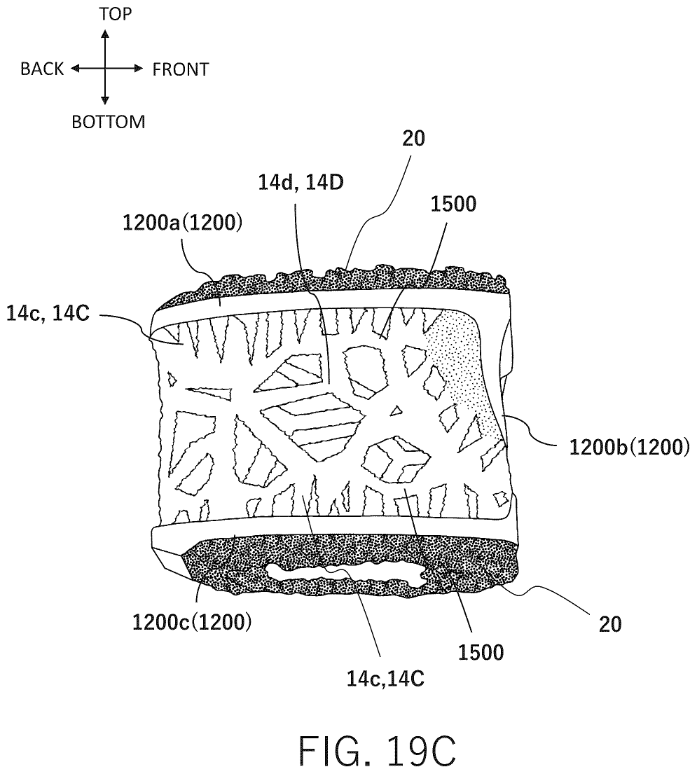

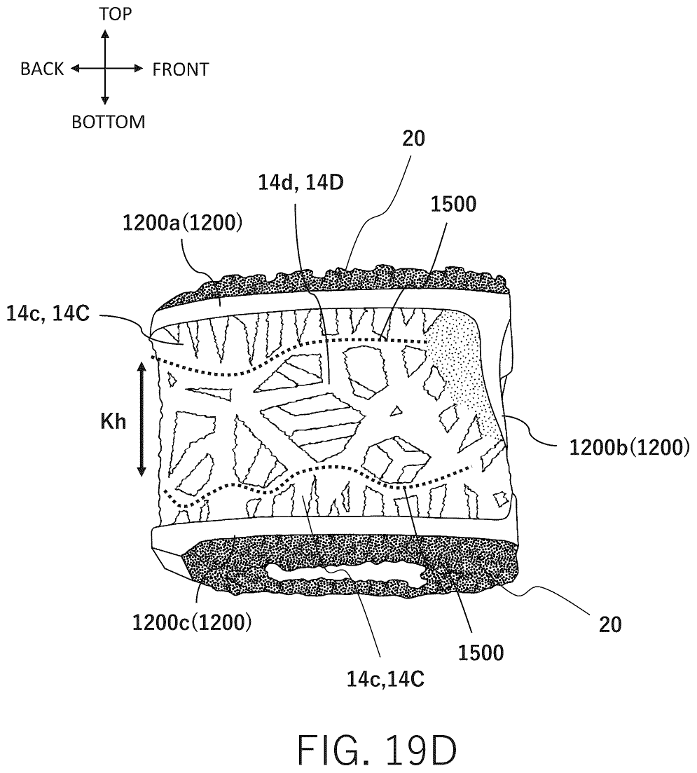

The implant device 1000 has parallel struts 1500 between the plurality of first struts 14C and the plurality of second struts 14D as shown in FIGS. 19A, 19B, 19C and 19D. The parallel struts 1500 extend along the solid surface 20 compared to the struts 14c, 14d and 14e. The parallel struts 1500 are connected to the struts 14c and 14d. The parallel strut 1500 may, for example, be thinner than struts 14d or thicker than the struts 14c. In this embodiment, the parallel struts 1500 are configured to be as thick as the struts 14d, and the parallel struts 1500 may be a part of the plurality of second struts 25.

Like the implant device 10, an outer surface 1200bbB of the screw holes 1200bb includes a region 1300 where the surface roughness is larger than the surface roughness of an inner surface 1200bbA. The outer surface 1200bbB is configured to connect with, for example, the struts 14c and/or struts 14d. The parallel struts 1500 extend from, for example, the outer surface 1200bbB of the screw hole 1200bb. The surface roughness of an outer surface of the parallel struts 1500 may be similar to that of the outer surface 1200bbB.

The parallel struts 1500 may bent up and down along the length. In this embodiment, the implant device 1000 may include two parallel struts 1500 on the top and bottom sides. The parallel struts 1500 on top side and bottom sides are arranged along each other to hold, for example, a roughly constant interval Kh. For example, when the parallel strut 1500 of top side bends downward, the corresponding part of the parallel strut 1500 of bottom side bends downward, and the approximately constant interval Kh is maintained along the length. The interval Kh may be, for example, exactly the same or different along the length. The interval Kh is configured to be less than, for example, an interval between the parallel struts 1500 of the top side and the solid surface 1200a and/or an interval between the parallel struts 1500 of the bottom side and the solid surface 1200c.

The implant device 1000 may include the parallel struts 1500 from the left side to the right side, or on a part of the left side, rear side and/or right side. The struts 14c between the parallel struts 1500 and the solid surface 1200, which is positioned on the top side and on the bottom side, near the parallel struts 1500 may extend along the vertical direction of the solid surface 1200. The longest strut in the struts 14c of the top side, which is located between the solid surface 1200 of the top side and the parallel struts 1500, and the longest strut in the struts 14c of the bottom side, which is located between the solid surface 1200 of the bottom side and the parallel struts 1500, are located so as to deviate in lateral direction (the direction in which the parallel struts 1500 extend).

The foregoing descriptions are examples and not limitations.

What is claimed is:

1. A surgical implant device, comprising:
a first solid surface;
a second solid surface opposing the first solid surface; and
a third solid surface connecting the first solid surface and the second solid surface, the third solid surface comprising:
a hole penetrating through the first solid surface and the second solid surface, the hole comprising:
an outer surface comprising a first region; and
an inner surface,
wherein a surface roughness of the first region is greater than a surface roughness of the inner surface.

2. The surgical implant device of claim 1, further comprising:
a lattice volume comprising a plurality of struts,
wherein the lattice volume is located between the first solid surface and the second solid surface.

3. The surgical implant device of claim 2, wherein a surface roughness of the outer surface is greater than a surface roughness of the plurality of struts.

4. The surgical implant device of claim 2, further comprising:
a porous surface located on at least one of a top side of the first solid surface and a bottom side of the second solid surface,
wherein the surface roughness of the first region is smaller than a surface roughness of the porous surface.

5. The surgical implant device of claim 2, further comprising:
a second region located on at least one of an underside of the first solid surface, behind the third solid surface and an upper side of the second solid surface,
wherein a surface roughness of the second region is greater than a surface roughness of the inner surface.

6. The surgical implant device of claim 5, further comprising:
a porous surface located on at least one of a top side of the first solid surface and a bottom side of the second solid surface,
wherein the surface roughness of the second region is smaller than a surface roughness of the porous surface.

7. The surgical implant device of claim 2, further comprising:
a first pore that is surrounded by the hole and the plurality of struts connected to the hole; and
a second pore that is surrounded only by the plurality of struts,
wherein the first pore is smaller than the second pore.

8. The surgical implant device of claim 2,
wherein the plurality of struts comprises a plurality of connecting struts that are connected to an outer surface of the lattice volume in a perpendicular direction to a length direction of the plurality of connecting struts.

9. The surgical implant device of claim 8, further comprising a recess configured by the outer surface and the plurality of connecting struts.

10. The surgical implant device of claim 8, further comprising:
a porous surface located on at least one of a top side of the first solid surface and a bottom side of the second solid surface, the porous surface comprising
a flat region on a lateral direction of the porous surface.

11. The surgical implant device of claim 10, wherein the flat region is coplanar with sides of at least one of the first solid surface, the second solid surface and the third solid surface.

12. The surgical implant device of claim 10, wherein the flat region constitutes surfaces inclining inwardly to sides of at least one of the first solid surface, the second solid surface and the third solid surface.

13. The surgical implant device of claim 2, further comprising:
at least one frontmost strut of the plurality of struts, the at least one frontmost strut comprising at least one flat portion that is flatter as compared to portions of inside struts of the plurality of struts located inside the at least one frontmost strut.

14. The surgical implant device of claim 13, wherein the at least one frontmost strut comprises a plurality of frontmost struts, and wherein the at least one flat region of each frontmost strut of the plurality of frontmost struts are coplanar.

15. The surgical implant device of claim 13, wherein the at least one flat region is coplanar with at least one of a side of the first solid surface, a front side of the third solid surface and/or a side of the second solid surface.

16. The surgical implant device of claim 2, wherein the plurality of struts are located inside an outer periphery of the first solid surface and the second solid surface.

17. The surgical implant device of claim 2, further comprising a parallel strut of the plurality of struts extending along the first solid surface or the second solid surface.

18. The surgical implant device of claim 17, wherein a width of the parallel strut is greater than a width of a given strut of the plurality of the struts.

19. The surgical implant device of claim 2, further comprising:
a first parallel strut of the plurality of struts located on a side of the first solid surface; and
a second parallel strut of the plurality of struts located on a side of the second solid surface.

20. The surgical implant device of claim 19, wherein an interval between the first parallel strut and the second strut is larger than an interval between the first parallel strut and the first solid surface or an interval between the second parallel strut and the second solid surface.
